SEQUENCE LISTING (8 sequences)

SEQ ID NO: 1 - protein, 180 aa, Mus musculus
  mat_peptide (1)..(180)
SPSAPVNVTVRHLKANSAVVSWDVLEDEVVIGFAISQQKKDVRMLRFIQEVNTTTRSCALWDLEEDTEYIVHVQAISIQGQSPASEPVLFKTPREAEKMASKNKDEVTMKEMGRNQQLRTGEVLIIVVVLFMWAGVIALFCRQYDIIKDNEPNNNKEKTKSASETSTPEHQGGGLLRSKI

SEQ ID NO: 2 - protein, 111 aa, Mus musculus
  PEPTIDE (1)..(111)
SPSAPVNVTVRHLKANSAVVSWDVLEDEVVIGFAISQQKKDVRMLRFIQEVNTTTRSCALWDLEEDTEYIVHVQAISIQGQSPASEPVLFKTPREAEKMASKNKDEVTMKE

SEQ ID NO: 3 - DNA, 630 bp, Mus musculus
  CDS (1)..(630)
atgcccccagggccgtgcgcctggccgccccgcgccgcgctccgcctgtggctaggctgcgtctgcttcgcgctggtgcaggcggacagcccctcagcccctgtgaacgtgaccgtccggcacctcaaggccaactctgccgtggtcagctgggatgtcctggaggatgaagtggtcattggctttgccatctctcagcagaagaaggatgtgcggatgctccggttcattcaggaggtgaacaccaccacccggtcctgcgctctctgggacctggaggaggacacagaatatatcgtccatgtgcaggccatctccatccagggacagagcccagccagtgagcctgtgctcttcaagaccccacgcgaggctgaaaagatggcctcaaagaacaaagatgaggtgaccatgaaggagatggggaggaaccagcagctgcgaacgggggaggtgctgatcattgttgtggtcctcttcatgtgggcaggtgttatagctctcttctgccgccagtatgatatcatcaaggacaacgagcccaataacaacaaggagaaaaccaagagcgcatcagaaaccagcacaccggagcatcagggtgggggtctcctccgcagcaagatatga

SEQ ID NO: 4 - DNA, 36 bp, Artificial Sequence
  Forward primer for PCR amplification of FNDC5.
atttgcggccgcggcccaggagtcgccatgccccca

SEQ ID NO: 5 - DNA, 34 bp, Artificial Sequence
  Reverse primer for PCR amplification of FNDC5.
ccgctcgagttatcttgctgcggaggagaccccc

SEQ ID NO: 6 - DNA, 333 bp, Mus musculus
  CDS (1)..(333)
agcccctcagcccctgtgaacgtgaccgtccggcacctcaaggccaactctgccgtggtcagctgggatgtcctggaggatgaagtggtcattggctttgccatctctcagcagaagaaggatgtgcggatgctccggttcattcaggaggtgaacaccaccacccggtcctgcgctctctgggacctggaggaggacacagaatatatcgtccatgtgcaggccatctccatccagggacagagcccagccagtgagcctgtgctcttcaagaccccacgcgaggctgaaaagatggcctcaaagaacaaagatgaggtgaccatgaaggag

SEQ ID NO: 7 - DNA, 25 bp, Artificial Sequence
  Forward primer for PCR amplification of irisin.
atttgtatacagcccctcagcccct

SEQ ID NO: 8 - DNA, 33 bp, Artificial Sequence
  Reverse primer for PCR amplification of irisin.
ccggagctctcactccttcatggtcacctcatc

(19) United States
(12) Patent Application Publication
(10) Pub. No.: US 2016/0256522 A1
(43) Pub. Date: Sep. 8, 2016

(54) METHOD FOR ENHANCING WOUND HEALING

(71) Applicant: NATIONAL SUN YAT-SEN UNIVERSITY, Kaohsiung city (TW)

(72) Inventors: Ming-Hong Tai, Kaohsiung city (TW); Shih-Wei Lin, Kaohsiung city (TW); Hsuan-Yi Hsiao, Kaohsiung city (TW); Chang-Yi Wu, Kaohsiung city (TW); Shih-Hsuan Cheng, Kaohsiung city (TW); Po-Han Tai, Kaohsiung city (TW); Han-En Tsai, Kaohsiung city (TW); Chia-Wen Hsieh, Kaohsiung city (TW); Jian-Ching Wu, Kaohsiung city (TW)

(73) Assignee: NATIONAL SUN YAT-SEN UNIVERSITY, Kaohsiung city (TW)

(21) Appl. No.: 14/846,891

(22) Filed: Sep. 7, 2015

(30) Foreign Application Priority Data
Mar. 5, 2015 (TW) 104107043

(51) Int. Cl. A61K 38/16

(52) U.S. Cl. CPC A61K 38/164

(57) ABSTRACT

The invention relates to a method for enhancing wound healing in a subject in need thereof, comprising administering to the subject a composition comprising fibronectin type III domain-containing protein 5 (FNDC5) or its cleaved fragment irisin in an amount effective to enhance wound healing

### CROSS-REFERENCES TO RELATED APPLICATIONS

The present application claims priority to Taiwan Patent Application No. 104107043 filed on Mar. 5, 2015, incorporated herein by reference in its entirety. The sequence listing text file, file name 2397-NCSU-US_SEQLIST.txt created Sep. 7, 2015, file size 9033 bytes, is incorporated herein by reference in its entirety.

### FIELD OF THE INVENTION

The invention relates to a method for enhancing wound healing. Particularly, the enhanced wound healing is through stimulation of individual angiogenesis, cell migration, and cell proliferation.

### BACKGROUND OF THE INVENTION

Myokine is a critical cytokine in organism which is major secreted from muscle cells after exercise. In 2012, Bostrom et al. found a new myokine called fibronectin type III domain-containing protein 5 (FNDC5) and its cleaved fragment, irisin (Nature. 2012 Jan. 11; 481 (7382): 463-8). Bostrom et al. also found that FNDC5 and its cleaved fragment, irisin, seem to drive browning of white fat. Further, FNDC5 or irisin potently increases energy expenditure, reduces body weight and alleviates diabetes. Functions of FNDC5 and its cleaved fragment, irisin, are similar.

The recent reports only point out that irisin enhances cells proliferation via the extracellular signal-related kinase (ERK) signaling pathway and protects the cell from high glucose-induced apoptosis (PLoS One. 2014 Oct. 22; 9(10):e110273). However, there is still no report about whether FNDC5 or irisin can modulate angiogenesis and wound healing.

Vascular endothelial growth factor (VEGF) is unique for its effects on multiple components of the wound healing cascade, including angiogenesis, cell migration, and recently shown epithelization and collagen deposition (Wound Repair Regen. 2013 November-December; 21(6):833-41). VEGF binds to VEGF receptor 1 (also called VEGFR1, VEGFR-1, or Flt-1) and VEGF receptor 2 (also called VEGFR2, VEGFR-2, or KDR) with high affinity. VEGFR-1 and VEGFR-2 are members of the Type III tyrosine kinase family, in which the signaling pathway of VEGFR-2 mediates the cell migration and proliferation (J Surg Res. 2009 May 15; 153(2):347-58).

### BRIEF DESCRIPTION OF THE DRAWINGS

FIG. 1A shows a schematic representation of fibronectin type III domain-containing protein 5 (FNDC5) and a cleaved fragment of FNDC5 (irisin) protein structure of the present invention, where C is a C-terminal domain; H is a hydrophobic domain; SP is a signal peptide.

FIG. 1B shows a schematic illustration of a signaling pathway and functions of FNDC5 in a cell.

FIG. 2 shows a result of a qualitative analysis of FNDC5 proteins, which are expressed in Escherichia coli and purified by the 6× Histidine (6×His) tag. The result was analyzed by a sodium dodecyl sulfate-polyacrylamide gel electrophoresis (SDS-PAGE) and a western blot assay. Lane A: Coomassie blue staining; Lane B: the western blot using an anti-FNDC5 antibody; Lane C: the western blot using an anti-6×His antibody; Lane D: Dithiothreitol (DTT) treatment with FNDC5 and the western blot using the anti-FNDC5 antibody.

FIG. 3A shows a result of the expression and stability of FNDC5 proteins at different temperatures. FNDC5 proteins were purified by nickel-nitrilotriacetic acid (Ni-NTA) beads binding the 6× Histidine tag and the stability of FNDC5 proteins storage at −80° C., −80° C. freeze and thaw (F&T), −20° C., 4° C., room temperature (RT, 25° C.), and 37° C. for 10 days. The result was analyzed by SDS-PAGE and a western blot assay. Upper panel: Coomassie blue staining; Bottom panel: the western blot using the anti-FNDC5 antibody.

FIG. 3B shows an effect of FNDC5 after incubation at different temperatures for 10 days on proliferation of endothelial cells. The proliferation of the endothelial cells was determined by a MMT assay and expressed as ratio to control. Data were mean±SEM of triplicates (*P<0.05, **P<0.01).

FIG. 4A shows an effect of FNDC5 on VEGF expression in endothelial cells. HUVECs were treated with FNDC5 (0.1, 1, and 10 ng/mL) for 24 h and subjected to a western blot analysis. Data were mean±SEM of triplicates (*P<0.05, **P<0.01).

FIG. 4B shows an effect of Avastin on FNDC5-modulated VEGF expression in endothelial cells. After adding Avastin (25 μg/mL), HUVECs were treated with FNDC5 (1 and 10 ng/mL) for 24 h and subjected to a western blot analysis. Data were mean±SEM of triplicates (*P<0.05, **P<0.01).

FIG. 5A shows an effect of FNDC5 on VEGFR2 phosphorylation in endothelial cells. HUVECs were treated with FNDC5 (0.1, 1, and 10 ng/mL) for 24 h and subjected to a western blot analysis. Data were mean±SEM of triplicates (*P<0.05, **P<0.01).

FIG. 5B shows an effect of Avastin on FNDC5-modulated VEGFR2/p-VEGFR2 expression in endothelial cells. After adding Avastin (25 μg/mL), HUVECs were treated with FNDC5 (1 and 10 ng/mL) for 24 h and subjected to a western blot analysis. Data were mean±SEM of triplicates (*P<0.05, **P<0.01).

FIG. 6A shows an effect of FNDC5 on Erk/p-Erk pathway in endothelial cells. HUVECs were treated with FNDC5 (0.1, 1, and 10 ng/mL) for 24 h and subjected to a western blot analysis. Data were mean±SEM of triplicates (*P<0.05, **P<0.01).

FIG. 6B shows an effect of Avastin on FNDC5-modulated VEGFR2/p-VEGFR2 expression in endothelial cells. After adding Avastin (25 μg/mL), HUVECs were treated with FNDC5 (1 and 10 ng/mL) for 24 h and subjected to a western blot analysis. Data were mean±SEM of triplicates (*P<0.05, **P<0.01).

FIG. 7A shows an effect of FNDC5 on p38 mitogen-activated protein kinase (p38 MAPK)/p-p38 MAPK pathway in endothelial cells. HUVECs were treated with FNDC5 (0.1, 1, and 10 ng/mL) for 24 h and subjected to a western blot analysis. Data were mean±SEM of triplicates (*P<0.05, **P<0.01).

FIG. 7B shows an effect of Avastin on FNDC5-modulated p38 MAPK/p-p38 MAPK expression in endothelial cells. After adding Avastin (25 μg/mL), HUVECs were treated with FNDC5 (1 and 10 ng/mL) for 24 h and subjected to a western blot analysis. Data were mean±SEM of triplicates (*P<0.05, **P<0.01).

FIG. 8A shows an effect of FNDC5 on Akt/p-Akt pathway in endothelial cells. HUVECs were treated with FNDC5 (0.1, 1, and 10 ng/mL) for 24 h and subjected to a western blot analysis. Data were mean±SEM of triplicates (*P<0.05, **P<0.01).

FIG. 8B shows an effect of Avastin on FNDC5-modulated Akt/p-Akt expression in endothelial cells. After adding Avastin (25 μg/mL), HUVECs were treated with FNDC5 (1 and 10 ng/mL) for 24 h and subjected to a western blot analysis. Data were mean±SEM of triplicates (*P<0.05, **P<0.01).

FIG. 9A shows an effect of FNDC5 on eNOS/p-eNOS and iNOS protein level in endothelial cells. HUVECs were treated with FNDC5 (0.1, 1, and 10 ng/mL) for 24 h and subjected to a western blot analysis. Data were mean±SEM of triplicates (*p<0.05, **P<0.01).

FIG. 9B shows an effect of Avastin on FNDC5-modulated eNOS/p-eNOS and iNOS expression in endothelial cells. After adding Avastin (25 μg/mL), HUVECs were treated with FNDC5 (1 and 10 ng/mL) for 24 h and subjected to a western blot analysis. Data were mean±SEM of triplicates (*P<0.05, **P<0.01).

FIG. 10 shows an effect of Avastin on FNDC5-modulated NFκB p105, NFκB p65, and NFκB p50 expression in endothelial cells. After adding Avastin (25 μg/mL), HUVECs were treated with FNDC5 (1 and 10 ng/mL) for 24 h and subjected to a western blot analysis. Data were mean±SEM of triplicates (*P<0.05, **P<0.01).

FIG. 11 shows an effect of FNDC5 on HIF-1α pathway in endothelial cells. HUVECs were treated with FNDC5 (0.1, 1, and 10 ng/mL) for 24 h and subjected to a western blot analysis. Data were mean±SEM of triplicates (*P<0.05, **P<0.01).

FIG. 12 shows an effect of FNDC5 on proliferation of endothelial cell. HUVEC (3×10³ cells/well) were treated with FNDC5 (0.1, 1, and 10 ng/mL) for 24 hours. Moreover, VEGF (10 ng/mL) was included as a positive control. The proliferation of HUVEC was determined by MMT assay and expressed as ratio to control. Data were mean±SEM of triplicates (*P<0.05, **P<0.01).

FIG. 13A shows a function of FNDC5 effect on scratch wound healing assay on 1% serum in endothelial cells. The profile of wound healing assay were shown (10× magnification) in endothelial cells treated with FNDC5 proteins (10 ng/mL). Moreover, VEGF (10 ng/mL) was included as a positive control.

FIG. 13B shows a function of FNDC5 effect on scratch wound healing assay on 1% serum in endothelial cells. The endothelial cells were treated with FNDC5 (10 ng/mL). Moreover, VEGF (10 ng/mL) was included as a positive control. Quantification of migrated cells were counted at high power fields and expressed as mean±SEM of triplicates. (*p<0.05, **p<0.001 ratio to control).

FIG. 14A shows an effect of FNDC5 on migration of endothelial cells. The profile of migration in endothelial cells were treated with FNDC5 proteins (0.1, 1, 10 ng/mL) and positive control VEGF (10 ng/mL) on polycarbonate membrane and membrane were stained with dye.

FIG. 14B shows an effect of FNDC5 on migration of the endothelial cells. Quantification of the cells were counted at high power fields and expressed as mean±SEM of triplicates. (*p<0.05, **p<0.001 ratio to control).

FIG. 14C shows an effect of irisin on migration of the endothelial cells. The profile of migration in endothelial cells were treated with irisin proteins (1, 10, 100 ng/mL) on polycarbonate membrane and membrane were stained with dye.

FIG. 14D shows an effect of irisin on migration of the endothelial cells. Quantification of the cells were counted at high power fields and expressed as mean±SEM of triplicates. (*p<0.05, **p<0.001 ratio to control).

FIG. 15A shows an effect of FNDC5 on tube formation in endothelial cells. The profile of tube formation in HUVEC cells were treated with FNDC5 proteins (0.1, 1, 10 ng/mL) and positive control VEGF (10 ng/mL) on Matrigel coated plate. The tubular structure was monitored and recorded under light microscopy.

FIG. 15B shows an effect of FNDC5 on tube formation in endothelial cells. Tube formation was quantified by counting the number of rings. Data were expressed as mean±SEM of triplicates. (*p<0.05, **p<0.001 ratio to control)

FIG. 15C shows an effect of FNDC5 and irisin on tube formation in endothelial cells. The profile of tube formation in HUVEC cells were treated with FNDC5 proteins (10 ng/mL), irisin proteins (10 ng/mL), and positive control VEGF (10 ng/mL) on Matrigel coated plate. The tubular structure was monitored and recorded under light microscopy.

FIG. 15D shows an effect of FNDC5 and irisin on tube formation in endothelial cells. Tube formation was quantified by counting the number of rings. Data were expressed as mean±SEM of triplicates. (*p<0.05, **p<0.001 ratio to control)

FIG. 16A shows an effect of FNDC5 on microvessel sprouting in aorta rings. Rat aortic rings placed in Matrigel were treated with phosphate-buffered saline (PBS, control group), FNDC5 (0.1, 1, and 10 ng/mL), and positive control PDGF (10 ng/mL) and a vessel sprout from various aorta samples was observed on day 7. n=6; Scale bar=100 μm.

FIG. 16B shows an effect of FNDC5 on microvessel sprouting in aorta rings. Quantification analysis of the new blood vessel growth in a defined area was performed mean±SD. Bars=3 mm. n=6 per group. (*, p<0.05 and **, p<0.01.)

FIG. 16C shows an effect of FNDC5 and irisin on microvessel sprouting in aorta rings. Rat aortic rings placed in Matrigel were treated with PBS, FNDC5 (1, 10, and 100 ng/mL), irisin (1, 10, and 100 ng/mL) and positive control VEGF (10 ng/mL) and a vessel sprout from various aorta samples was observed on day 7. n=6; Scale bar=100 μm.

FIG. 16D shows an effect of FNDC5 and irisin on microvessel sprouting in aorta rings. Quantification analysis of the new blood vessel growth in a defined area was performed mean±SD. Bars=3 mm. n=6 per group. (*, p<0.05 and **, p<0.01.)

FIG. 17A shows an effect of FNDC5 on angiogenesis in a transgenic Tg(fli-1: EGFP)^{y1} zebrafish larva. The Tg(fli-1: EGFP)^{y1} zebrafish larva were treated with FNDC5 (10 ng/mL) and positive control VEGF (10 ng/mL) at 6 hpf (n=12 per group) then monitored for imaging recording at various time intervals. At 48 hpf, the representative photographs of intersegmental vessels (ISV) fluorescence in Tg(fli-1: EGFP)^{y1} zebrafish treated with FNDC5 (10 ng/mL) and positive control VEGF (10 ng/mL) were shown (40× magnification; Scale bars, 100 μm). The ISV fluorescence were quantified and expressed as mean±SD percentages of control (n=12).

FIG. 17B shows an effect of FNDC5 on angiogenesis in a transgenic Tg(fli-1: EGFP)^{y1} zebrafish larva. The Tg(fli-1: EGFP)^{y1} zebrafish larva were treated with FNDC5 (10 ng/mL) and positive control VEGF (10 ng/mL) at 6 hpf (n=12 per group) then monitored for imaging recording at various time intervals. At 48 hpf, the representative photographs of subintestinal vessel plexus (SIV) fluorescence in Tg(fli-1: EGFP)^{y1} zebrafish treated with FNDC5 (10 ng/mL) were analyzed (100× magnification; Scale bars=100 μm). Arcades in the vesicle-like structure of SIV were quantified and expressed as mean±SD (n=12).

FIG. 17C shows an effect of FNDC5 on angiogenesis in a transgenic Tg(fli 1a-nEGFP)^{y7} zebrafish larva. The Tg(fli 1a-nEGFP)^{y7} zebrafish larva were treated with FNDC5 (10 ng/mL) at 6 hpf (n=7 per group) then monitored for imaging recording at various time intervals. At 24 hpf, the representative photographs of subintestinal vessel plexus (SIV) fluorescence in Tg(fli 1a-nEGFP)^{y7} zebrafish treated with FNDC5 (10 ng/mL) were analyzed (400× magnification; Scale bars=100 μm). Arcades in the vesicle-like structure of SIV were quantified and expressed as mean±SD (n=7).

FIG. 18A shows an effect of FNDC5 on diabetic SD rat punch wound healing. Two groups of wounded diabetic SD rat (STZ induced) were treated every two days with PBS (control group) and FNDC5 protein (1 mg/mL) alone. Representative wounds at days 0, 4 and 8, 12 and 16 were shown.

FIG. 18B shows an effect of FNDC5 on diabetic SD rat punch wound healing. Two groups of wounded diabetic SD rat (STZ induced) were treated every two days with PBS (control group) and FNDC5 protein (1 mg/mL) alone. H&E stained images of skin were shown.

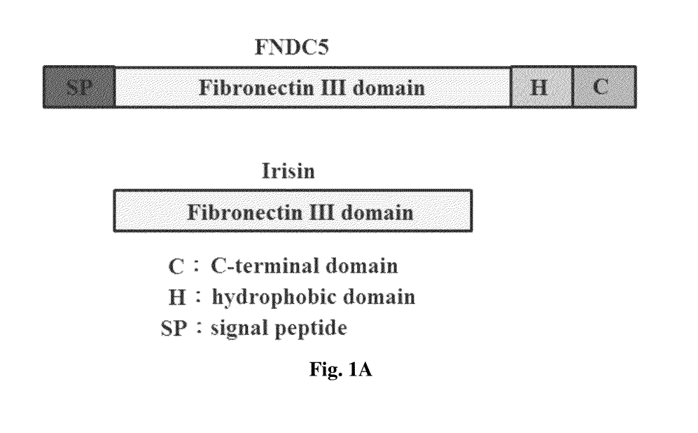

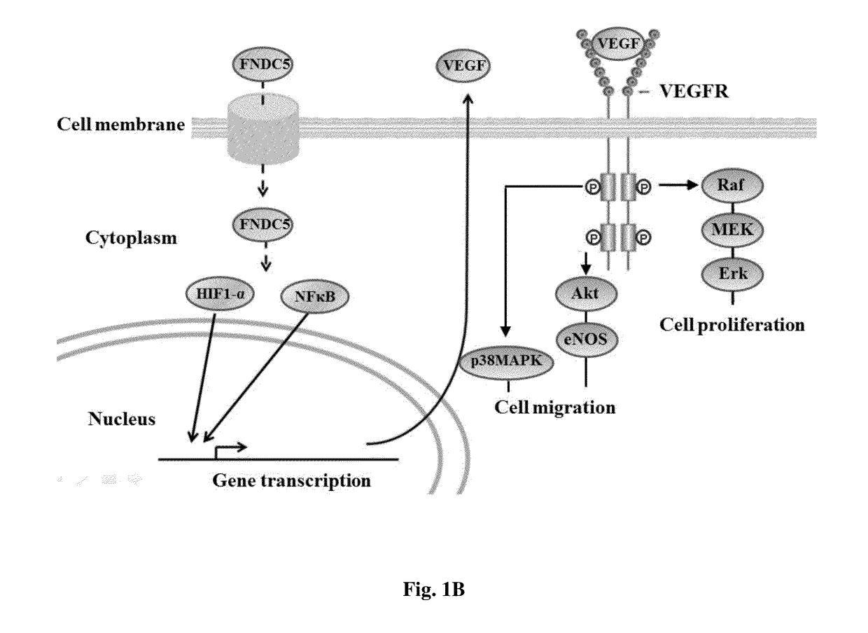

### SUMMARY OF THE INVENTION

The present invention is to provide a method for enhancing wound healing in a subject in need thereof, comprising administering to the subject a composition comprising fibronectin type III domain-containing protein 5 (FNDC5) or its cleaved fragment irisin in an amount effective to enhance wound healing.

### DETAILED DESCRIPTION OF THE INVENTION

The following definitions are offered for purposes of illustration, not limitation, in order to assist with understanding the discussion that follows.

As used herein, the term “a” or “an” are employed to describe elements and components of the invention. This is done merely for convenience and to give a general sense of the invention. This description should be read to include one or at least one and the singular also includes the plural unless it is obvious that it is meant otherwise.

As used herein, the term “or” is employed to describe “and/or”.

The present invention provides a method for enhancing wound healing in a subject, comprising the step of contacting a wound site with a composition comprising fibronectin type III domain-containing protein 5 (FNDC5) or its cleaved fragment irisin in an amount effective to enhance wound healing.

In a specific embodiment of the present invention, the amino acid sequence of the FNDC5 is SEQ ID NO: 1, and the amino acid sequence of irisin is SEQ ID NO: 2.

In a specific embodiment of the present invention, the fibronectin type III domain-containing protein 5 is a recombinant protein expressed by an expression host. In a preferred embodiment of the present invention, the recombinant FNDC5 protein has a molecular weight from 32 kDa to 25 kDa. In a more preferred embodiment of the present invention, the recombinant FNDC5 protein has a molecular weight of 25 kDa.

In another specific embodiment of the present invention, irisin is a recombinant protein expressed by an expression host. In a preferred embodiment of the present invention, the recombinant irisin protein has a molecular weight of 12 kDa.

In a preferred embodiment of the method of the invention, the wound healing is enhanced by stimulation of angiogenesis, cell migration, and cell proliferation in the subject.

In a specific embodiment, FNDC5 of the present invention is stable at different temperatures. In a preferred embodiment, the recombinant FNDC5 can be stocked at a temperature ranges from −80° C. to 25° C. and maintain high stability and activity for at least 10 days.

In a specific embodiment of the present invention, FNDC5 induces a VEGF protein level in a dose-dependent manner. In another specific embodiment, FNDC5 restores the VEGF protein level which is inhibited by a VEGF-neutralizer. In a preferred embodiment, FNDC5 restores the VEGF protein level which is inhibited by Avastin.

In a specific embodiment of the present invention, FNDC5 elevates a VEGF protein level. The binding of VEGF and VEGFR induces phosphorylated activation of extracellular signal-related kinase (ERK), p38 mitogen-activated protein kinase (p38 MAPK), or protein kinase B (Akt/PKB) in the downstream signaling pathway, thereby induces biological response of the cells. In a preferred embodiment, the VEGFR is a VEGFR2.

In another specific embodiment, FNDC5 restores the VFGFR2 proteins level, the VFGFR2 proteins phosphorylation level, and the downstream signaling pathway proteins phosphorylation levels of the VEGFR2 which are inhibited by the VEGF-neutralizer. In a preferred embodiment, the VEGF-neutralizer is Avastin.

In a specific embodiment of the present invention, FNDC5 does not affect endothelial nitric oxide synthase (eNOS) proteins level, the eNOS proteins phosphorylation level, and inducible nitric oxide synthase (iNOS) proteins level in endothelial cells.

In another specific embodiment, FNDC5 restores the eNOS proteins phosphorylation level, eNOS proteins level, and the iNOS proteins level, and the downstream signaling pathway proteins phosphorylation levels of the VEGFR2 which are inhibited by the VEGF-neutralizer. In a preferred embodiment, the VEGF-neutralizer is Avastin.

In a specific embodiment of the present invention, FNDC5 elevates NFκBp105, NFκBp65, and NFκBp50 protein levels in endothelial cells.

In another specific embodiment, FNDC5 restores the NFκBp105, NFκBp65, and NFκBp50 protein levels which are inhibited by the VEGF-neutralizer. In a preferred embodiment, the VEGF-neutralizer is Avastin.

In a specific embodiment of the present invention, FNDC5 induces VEGF expression by inducing expression of HIF-1α, which is a member in the upstream signaling pathway of VEGF.

In a specific embodiment of the present invention, FNDC5 induces scratch wound healing of cells. In a preferred embodiment, FNDC5 induces scratch wound healing of the endothelial cells.

In a specific embodiment of the present invention, FNDC5 induces proliferation of cells. In a preferred embodiment, FNDC5 induces proliferation of the endothelial cells.

In a specific embodiment of the present invention, FNDC5 or irisin enhances proliferation, migration, and tube formation of the cells. In a preferred embodiment, the cells are the endothelial cells.

In a specific embodiment of the present invention, FNDC5 or irisin enhances the vessels outgrowth in organotypic aorta cultures.

In a specific embodiment of the present invention, FNDC5 induces vascular development in a subject. In one preferred embodiment, FNDC5 induces intersegmental vascular development in a larva of a transgenic zebrafish, Tg (fli-1 EGFP)^{y1}. In another preferred embodiment, FNDC5 induces subintestinal vascular development in the larva of the transgenic zebrafish, Tg (fli-1: EGFP)^{y1}. In the other preferred embodiment, FNDC5 induces subintestinal vascular development in a larva of transgenic zebrafish, Tg (fli 1a-nEGFP)^{y7}.

In a specific embodiment of the present invention, FNDC5 enhances wound healing in the subject. In a preferred embodiment, FNDC5 speeds up the incision wound healing in a diabetic rat.

The method of the invention can be applied to enhancing wound healing, wherein the wound is selected from incisions, lacerations, abrasions, puncture wounds, diabetes ulcers, a burn (resulted from fire, heat, radiation, electricity, caustic chemicals, or dermatological surgery), blisters, skin tears, donor or graft sites, acnes, contusions, hematoma, crushing injuries or injuries caused by dermabrasion or laser resurfacing.

The method of the invention can be used in the subject with diabetes mellitus. In a preferred embodiment, the patient suffers from diabetes ulcers.

The method of the invention can also be applied when the wound is a burn resulted from fire, heat, radiation, electricity, caustic chemicals, or dermatological surgery.

The method of the invention can be applied to wound healing or reconstructive surgery.

In a preferred embodiment, the composition of the invention is in a form selected from gel, cream, paste, lotion, spray, suspension, solution, dispersion salve, hydrogel or ointment formulation. In a more preferred embodiment, the composition is administered to the patient in need of such treatment by applying onto skin, injection or electroporation.

In a preferred embodiment of the present invention, the expression host is a bacterium, yeast, an insect cell, virus, or a mammalian cell. In a more preferred embodiment of the present invention, the bacterium is Escherichia coli.

In conclusion, FNDC5 or irisin can effectively enhance the rate of wound healing.

### TERM DEFINITION

In accordance with the present invention there may be employed conventional molecular biology, microbiology, and recombinant DNA techniques within persons skilled in the art. Such techniques are explained fully in the literature.

“Wounds” can be characterized as open wounds and closed wounds. Open wounds can be classified into a number of different types, including incisions (caused by a clean, sharp-edged object such as a knife or a razor), lacerations (rough, irregular wounds caused by crushing or ripping forces), abrasions or grazes (a superficial wound in which the topmost layers of the skin are scraped off, often caused by a sliding fall onto a rough surface), and puncture wounds (caused by an object puncturing the skin, such as a nail or needle). Closed wounds have far fewer categories, but are just as dangerous as open wounds. They are contusions or bruise (caused by blunt force trauma that damages tissues under the skin), hematoma (caused by damage to a blood vessel that in turn causes blood to collect under the skin) and crushing injuries (caused by a great or extreme amount of force applied over a long period of time).

As used herein “burn” is the injury resulting from exposure to heat, electricity, radiation (for example, sunburn and laser surgery), caustic chemicals, or dermatological surgery.

As used herein, the term “sample” is selected from the group consisting of a tissue sample, a fecal sample, a urine samples, a cell homogenate, a blood sample, one or more biological fluids, or any combinations thereof.

As used herein, the term “subject” is meant any animal, including, without limitation, humans such as dogs, cats, mice, rats, cattle, sheep, pigs, goats, and non-human primates. In more preferred embodiments, the subject is a human.

As used herein, the term “host” is a bacterium, a yeast, an insect cell or a mammalian cell. More preferably, the bacterium is Escherichia coli.

The terms used in the description herein will have their ordinary and common meaning as understood by those skilled in the art, unless specifically defined otherwise.

### EXAMPLES

The examples below are non-limiting and are merely representative of various aspects and features of the present invention.

All values in the examples were expressed as means±standard deviation (SD) or means±standard error of the mean (SEM). A paired t test was statistically assessed to evaluate the differences between the groups. The differences were considered to be statistically significant when p<0.05

Human umbilical vein endothelial cells (HUVECs) were isolated from umbilical veins and cultured in M199 medium (Life Technologies, Gaithersburg, Md.) as previously described (Atherosclerosis. 2012 April; 221(2):341-9, Exp Biol Med (Maywood). 2006 June; 231(6):782-8, Arch Biochem Biophys. 2012 Mar. 1; 519(1):8-16, and Eur J Clin Invest. 2000 July; 30(7):618-29). HUVECs were used for all the examples at adjust to a final concentration of 20% total protein in fetal bovine serum (FBS) and 2 mM L-glutamine under humidified conditions in 95% air and 5% CO₂ at 37° C.

### Example 1

### Methods

### Cloning, Expression, and Purification of Recombinant FNDC5

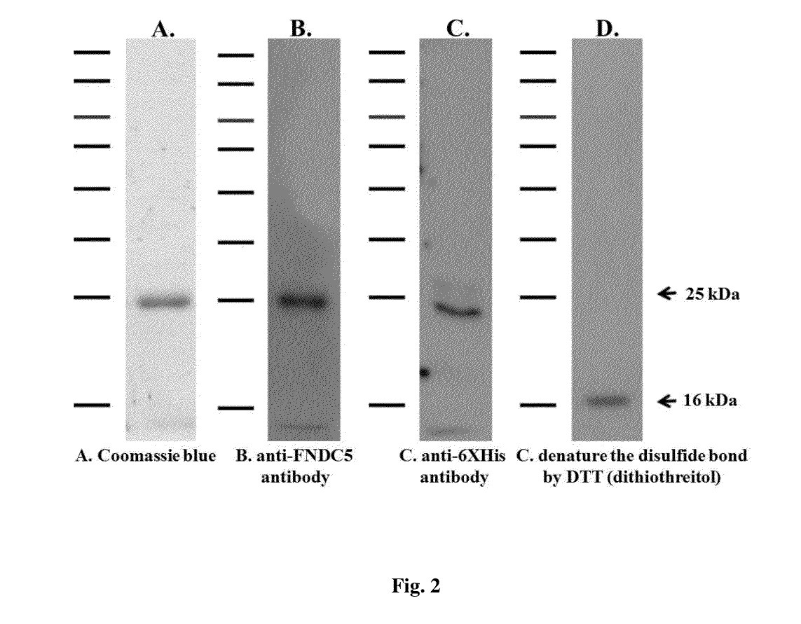

The DNA sequence of SEQ ID NO: 3 encoding fibronectin type III domain-containing protein 5 (FNDC5) was amplified by polymerase chain reaction (PCR) with forward primer of SEQ ID NO: 4 and reverse primer of SEQ ID NO: 5, and then subcloned into the NotI and BamHI sites of the pET28a vector (Novagen Inc., Madison, Wis.) to yield the pET28a-FNDC5 plasmid. For expression and purification, the pET28a-FNDC5 plasmid was transformed into BL-21 (DE3) pLysS competent cells, which was derived from Escherichia coli (E. coli). Isopropyl-β-D-thiogalactopyranoside (IPTG) was used to induce the expression of the N-terminal extracellular domain (N-terminal ECD). The transformed cells were grown at 37° C. optical density (OD) 600 nm of 0.6-0.8. Subsequently, the cells were added IPTG to final concentration (1 mM) and incubated for 4 hours at 30° C. to induce the protein expression. The cell pellet was harvested by centrifugation at 6000 revolution per minute (rpm) for 5 minutes at 4° C., resuspended in lysis buffer (20 mM phosphate buffer at pH 8.0.20 mM imidazole, 150 mM NaCl, 1 mM EDTA, and containing 1 mM PMSF, 1 μg/mL aprotinin, 1 μg/mL leupeptin, and 1 μg/mL pepstatin), and then homogenized by sonication and centrifugation 9000 rpm for 30 minutes. The produced protein was purified with immobilized Ni²⁺ affinity chromatography and refolded by dialysis. After centrifugation, the supernatant was mixed with 1 mL nickel-nitrilotriacetic acid (Ni-NTA) beads for 20 minutes. After washing the beads, the recombinant protein was eluted with elution buffer (20 mM phosphate buffer, 250 mM imidazole, and 150 mM NaCl, pH 7.4). The Protein expression was detected from sodium dodecyl sulfate-polyacrylamide gel electrophoresis (SDS-PAGE) (Coomassie Blue staining) tests. After electrophoresis, the gels were stained in Coomassie blue R-250 reagent solution (50% methanol and 10% acetic acid) for 1 hour and destained with destain solution (10% acetic acid and 20% methanol). The purity of FNDC5 proteins were analyzed by Coomassie blue staining as a protein with a molecular weight of 25 kDa (FIG. 2, Lane A). The identity of recombinant FNDC5 was confirmed by a Western blot assay using anti-FNDC5 (FIG. 2, Lane B) and anti-6×His (FIG. 2, Lane C), respectively.

### Cloning, Expression, and Purification of Recombinant Irisin

The DNA sequence of SEQ ID NO: 6 encoding irisin was amplified by polymerase chain reaction (PCR) with forward primer of SEQ ID NO: 7 and reverse primer of SEQ ID NO: 8, and then subcloned into the NdeI and XhoI. sites of the pET15b vector (Novagen Inc., Madison, Wis.) to yield the pET15b-Irisin plasmid. For expression and purification, the pET15b-Irisin plasmid was transformed into BL-21 (DE3) pLysS competent cells, which was derived from Escherichia coli (E. coli). Isopropyl-β-D-thiogalactopyranoside (IPTG) was used to induce the expression of the N-terminal extracellular domain (N-terminal ECD). The transformed cells were grown at 37° C. optical density (OD) 600 nm of (L6-0.8. Subsequently, the cells were added IPTG to final concentration (1 mM) and incubated for 4 hours at 30° C. to induce the protein expression. The cell pellet was harvested by centrifugation at (3000 revolution per minute (rpm) for 5 minutes at 4° C., resuspended in lysis buffer (20 mM phosphate buffer at pH 8.0.20 mM imidazole, 150 mM NaCl, 1 mM EDTA, and containing 1 mM PMSF, 1 μg/mL aprotinin, 1 μg/mL leupeptin, and 1 μg/mL pepstatin), and then homogenized by sonication and centrifugation 9000 rpm for 30 minutes. The produced protein was purified with immobilized Ni2+ affinity chromatography and refolded by dialysis. After centrifugation, the supernatant was mixed with 1 mL nickel-nitrilotriacetic acid (Ni-NTA) beads for 20 minutes, After washing the beads, the recombinant protein was eluted with elution buffer (20 mM phosphate buffer, 250 mM imidazole, and 150 mM NaCl, pH 7.4). The Protein expression was detected from sodium dodecyl sulfate-polyacrylamide gel electrophoresis (SDS-PAGE) (Coomassie Blue staining) tests. After electrophoresis, the gels were stained in Coomassie blue R-250 reagent solution (50% methanol and 10% acetic acid) for 1 hour and destained with destain solution (10% acetic acid and 20% methanol). The purity of irisin proteins were analyzed by Coomassie blue staining as a protein with a molecular weight of ˜12 kDa. The identity of recombinant irisin was confirmed by a Western blot assay using anti-FNDC5 and anti-6×His, respectively,

### Example 2

### Methods

### The Stability of Recombinant FNDC5 Proteins Storage at −80° C., −80° C. Freeze and Thaw, −20° C., 4° C., Room Temperature and 37° C. for 14 Days

To evaluate the stability of recombinant FNDC5 at different temperatures, recombinant FNDC5 solution (in phosphate buffered saline; 10 ng/mL) was placed at −80° C., −80° C. freeze and thaw, −20° C., 4° C., room temperature (RT, 25° C.), and 37° C. for 10 days then subjected to a SDS-PAGE/Western blot analysis and endothelial proliferation assay.

### Results

### The Recombinant FNDC5 Proteins were Stable at Different Temperatures

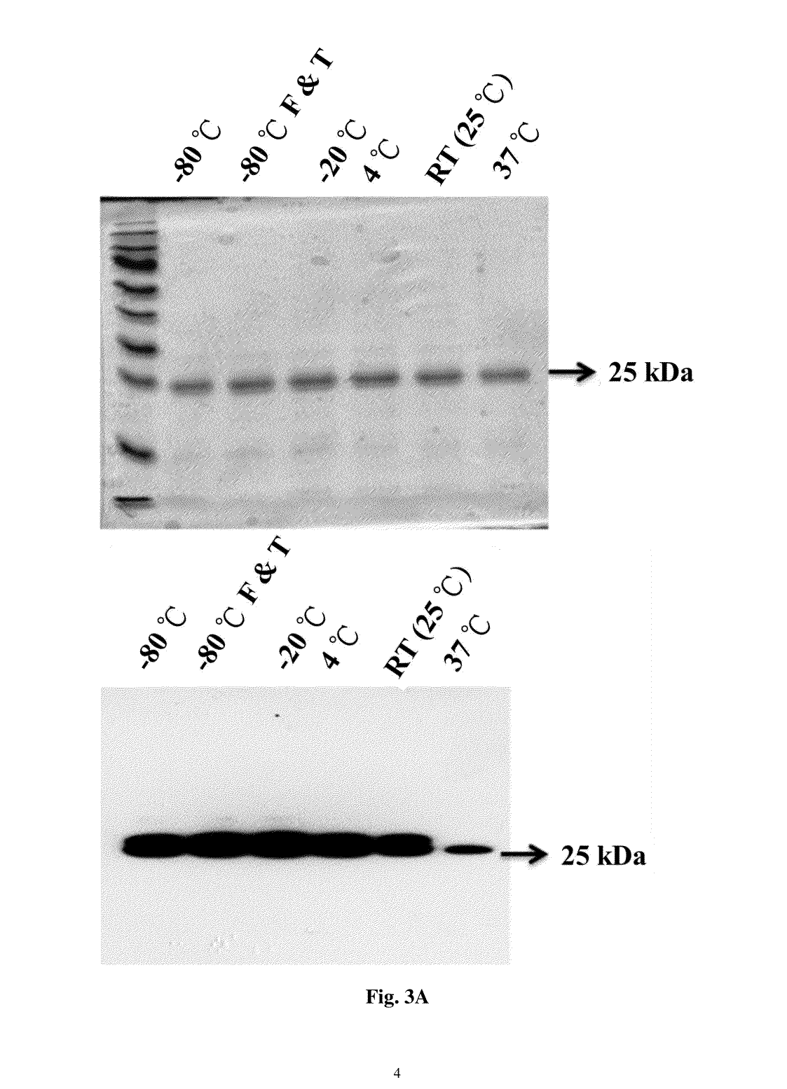

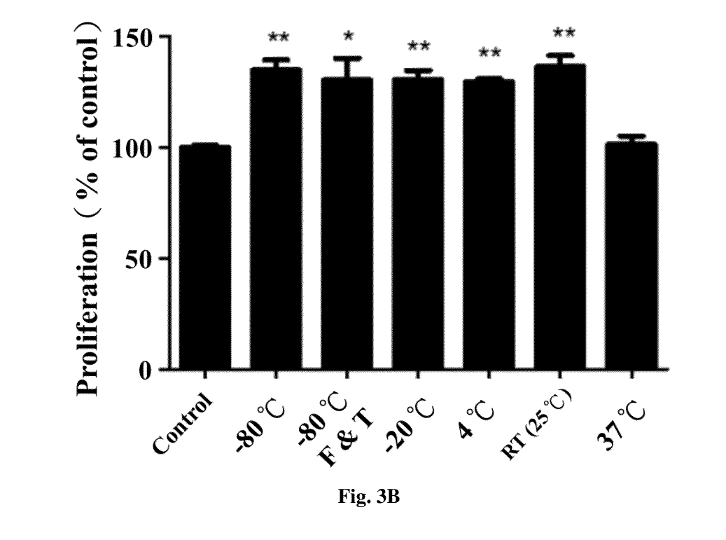

The recombinant FNDC5 was stable at room temperature for 10 days as no visible protein degradation from −80 to 25° C. (FIG. 3A) and 37° C. for 10 days that still induced the proliferation in endothelial cells by MTT assay (proliferation assay) (FIG. 3B). The Western result also showed the equal band in different condition (from −80 to 25° C.) except 37° C. FNDC5 had high stability at different temperatures.

### Example 3

### Methods

### Western Blotting and FNDC5 Induced VEGF Signaling Pathway

HUVEC lysates were prepared using RIPA lysis buffer (50 mM Tris-HCl pH 7.4, 1% NP-40, 0.25% sodium deoxycholate, 150 mM NaCl, 1 mM PMSF and protease inhibitors). An aliquot of proteins were separated by 10% SDS-PAGE and transferred onto the polyvinylidenedifluoride membranes (PVDF) (Immobilon-P membrane; Millipore, Bedford, Mass.). After blocking for 30 min, the membrane was incubated with primary antibodies for 2 hours at room temperature, and then conjugated with horseradish peroxidase (HRP)-conjugated secondary antibodies (anti-rabbit IgG, anti-mouse IgG or anti-goat; Santa Cruz, Burlingame Calif., USA) (1:5000 dilution) for 1 hour. Immunoreactivity was detected by ECL plus luminal solution (Amersham Biosciences, Piscataway, N.J., USA). The immunoband intensities were quantified by densitometric scanning. The primary antibodies used in the present invention were antibodies against FNDC5 (1:1000 dilution; abcam), His (1:500 dilution; Santa Cruz), VEGF (1:500 dilution; Santa Cruz), VEGFR2 (1:1000 dilution; EPICOMICS), p-VEGFR2 (1:500 dilution; Santa Cruz), Erk (1:1000 dilution; Cell Signaling), p-Erk (1:1000 dilution; Cell Signaling), p38 MAPK (1:1000 dilution; EPICOMICS), p-p38 MAPK (1:1000 dilution; EPICOMICS), Akt (1:500 dilution; Santa Cruz), p-Akt (1:500 dilution; Santa Cruz), eNOS (1:1000 dilution; BD), p-eNOS (1:1000 dilution; BD), iNOS (1:1000 dilution; BD), NFκB p105/50 (1:500 dilution; Santa Cruz), NFκB p65 (1:500 dilution; Santa Cruz), HIF1-α (1:1000 dilution), and β-actin (1:5000 dilution; SIGMA).

### Results

### FNDC5 Induces VEGF Protein Level Expression in Endothelial Cells

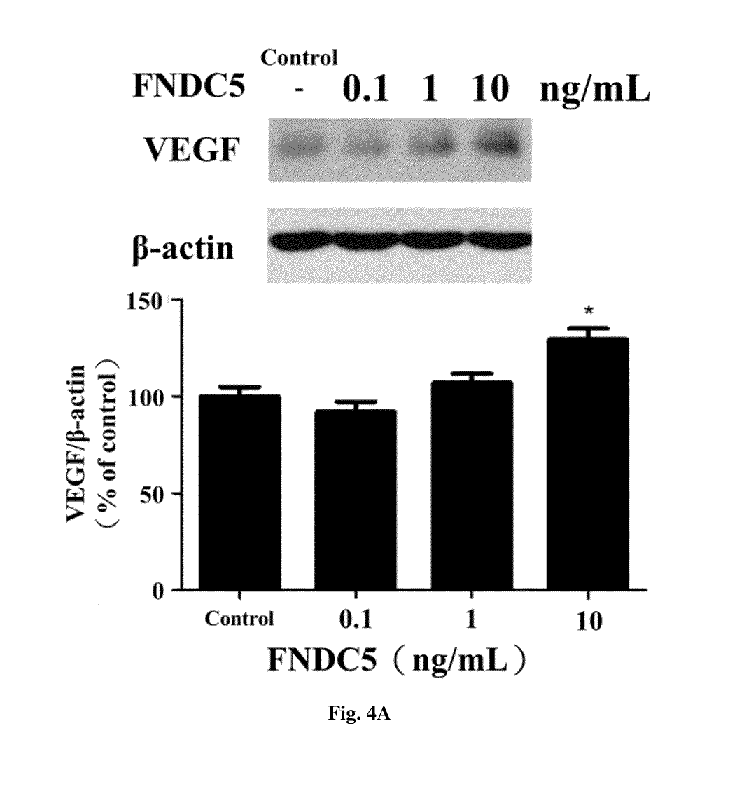

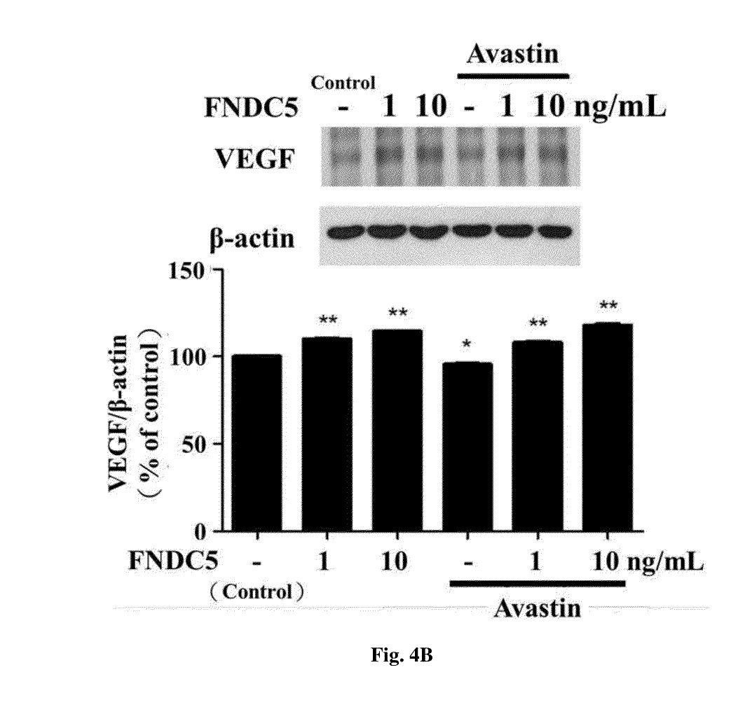

Because VEGF plays a pivotal role not only in angiogenesis but also wound healing (J Invest Dermatol. 2009 September; 129(9):2275-87), the present invention investigated the effect of FNDC5 on VEGF and downstream signaling pathway factors expression in the cells. The HUVECs were treated with FNDC5 (0.1, 1, and 10 ng/mL) for 24 h and subjected to a Western blot analysis. FIG. 4A showed that FNDC5 induced the VEGF protein level in a dose-dependent manner. Avastin was a monoclonal antibody that selectively conjugated to VEGF and then blocked its. Moreover, Avastin was used to trap VEGF. After adding Avastin (25 μg/mL), HUVECs were treated with FNDC5 (10 ng/mL) for 24 h and subjected to a Western blot analysis. FIG. 4B showed that FNDC5 restored the VEGF protein level.

### FNDC5 Elevated the VEGFR2 Expression and VEGFR2 Phosphorylation Protein Level in Endothelial Cells

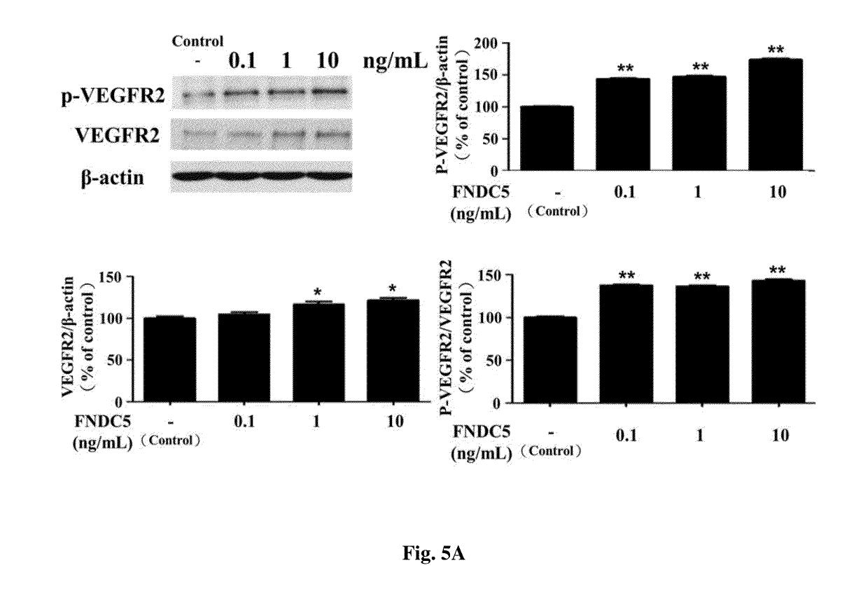

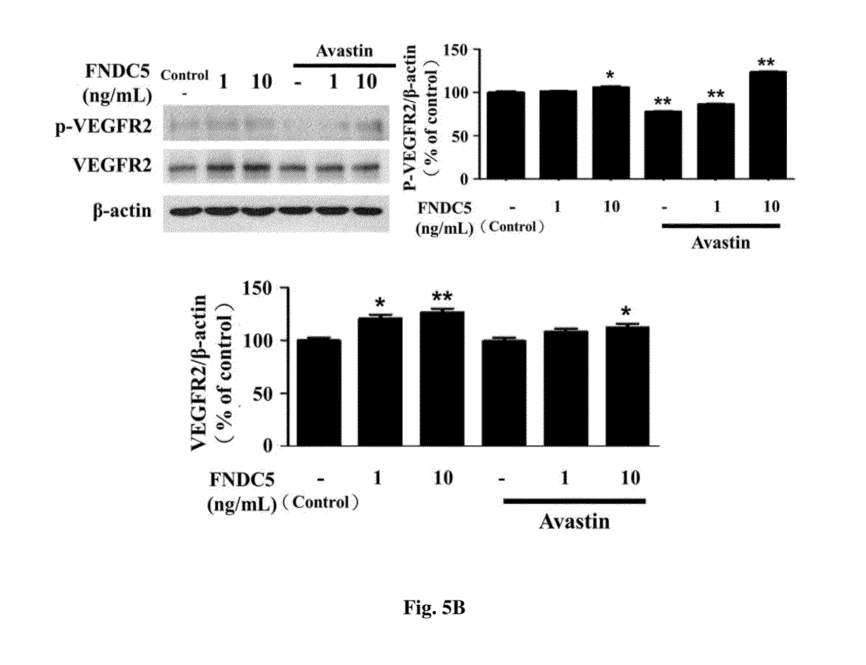

VEGFR2 is the main receptor mediating the function of VEGF in cells. Upon binding of VEGF to the VEGF receptor (VEGFR), dimerization and auto-phosphorylation of the intracellular receptor tyrosine kinases occurs. To evaluate the influence of FNDC5 on VEGFR2/p-VEGFR2 protein level in endothelial cells, HUVECs were treated with FNDC5 (0.1, 1, and 10 ng/mL) for 24 h and subjected to a Western blot analysis. The Western blot analysis revealed that FNDC5 induced the VEGFR2 expression in endothelial cells (FIG. 5A). Moreover, Avastin was used to inhibit VEGF confirmed the effect of FNDC5-modulated VEGFR2/p-VEGFR2 expression in endothelial cells. After adding Avastin (25 μg/mL), HUVECs were treated with FNDC5 (1 and 10 ng/mL) for 24 h and subjected to a Western blot analysis. FIG. 5B showed that FNDC5 (10 ng/mL) treatment restore the VEGFR2 and VEGFR2 phosphorylation expression in endothelial cells.

### FNSDC5 Regulated the VEGF/VEGFR2 Through the Erk/p38 MAPK Signal Pathway

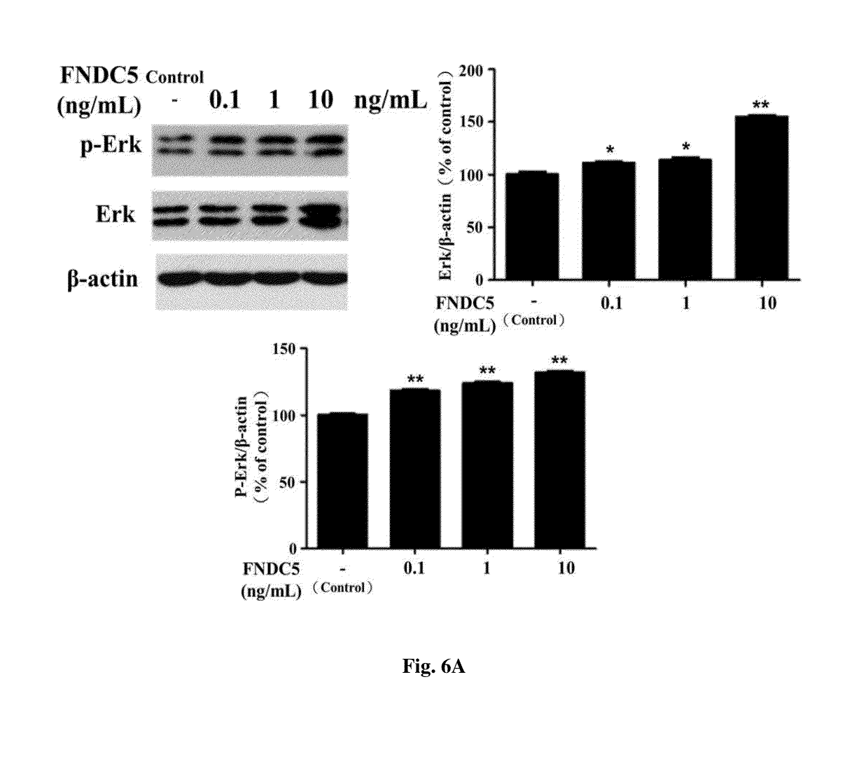

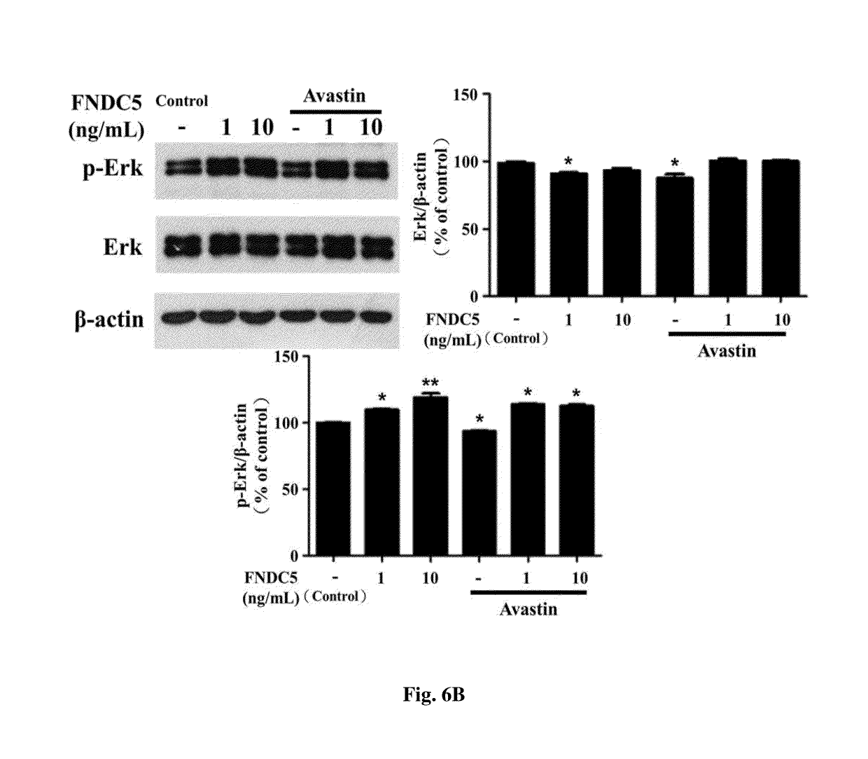

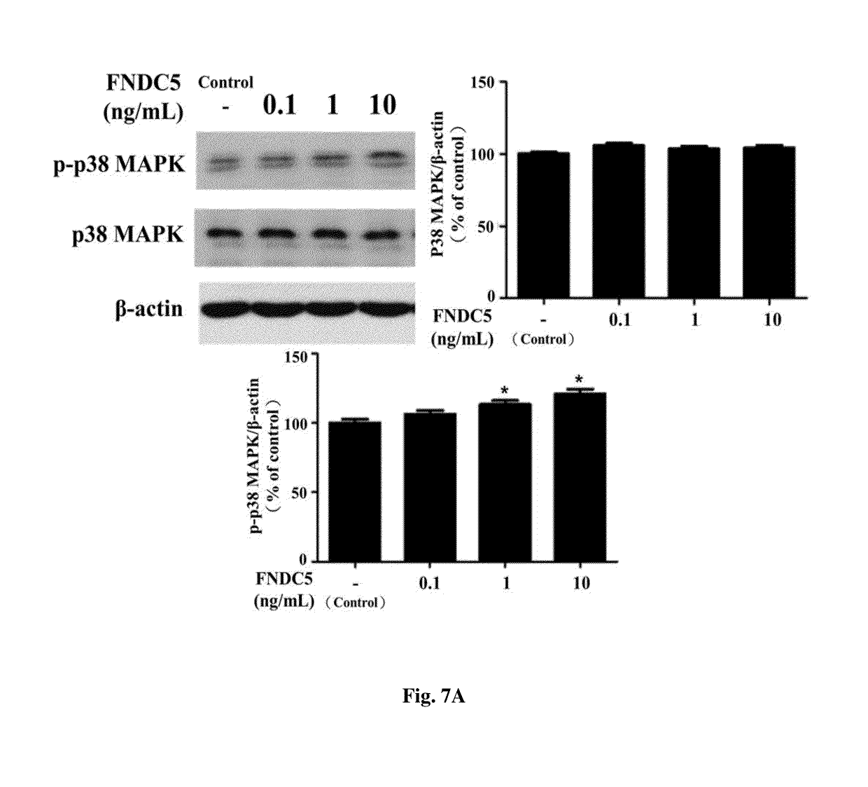

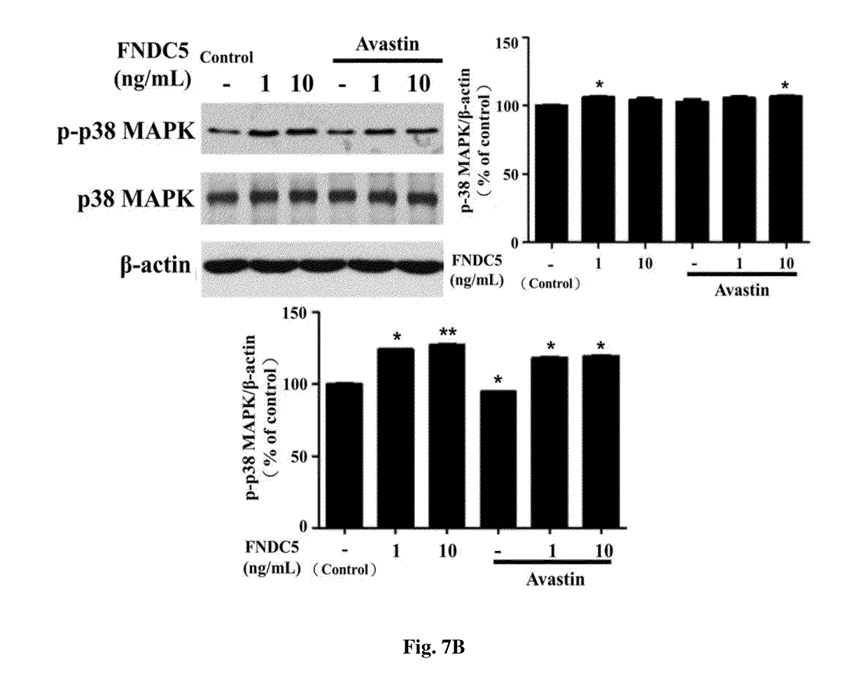

Upon binding of VEGF to the VEGF receptor (VEGFR), dimerization and auto-phosphorylation of the intracellular receptor tyrosine kinases occurs. Several downstream the protein like Erk, p38 MAPK and Akt pathways are activated, leading to biologic effects on the cells. To evaluate the influence of FNDC5 on downstream protein expression of VEGF, the western blot analysis revealed that FNDC5 induced the Erk and p38 MAPK expression in endothelial cells (FIG. 6A and FIG. 7A). Moreover, Avastin was used to inhibit VEGF confirmed that FNDC5 (1 and 10 ng/mL) could restore the Erk and p38 MAPK phosphorylation expression (FIG. 6B and FIG. 7B).

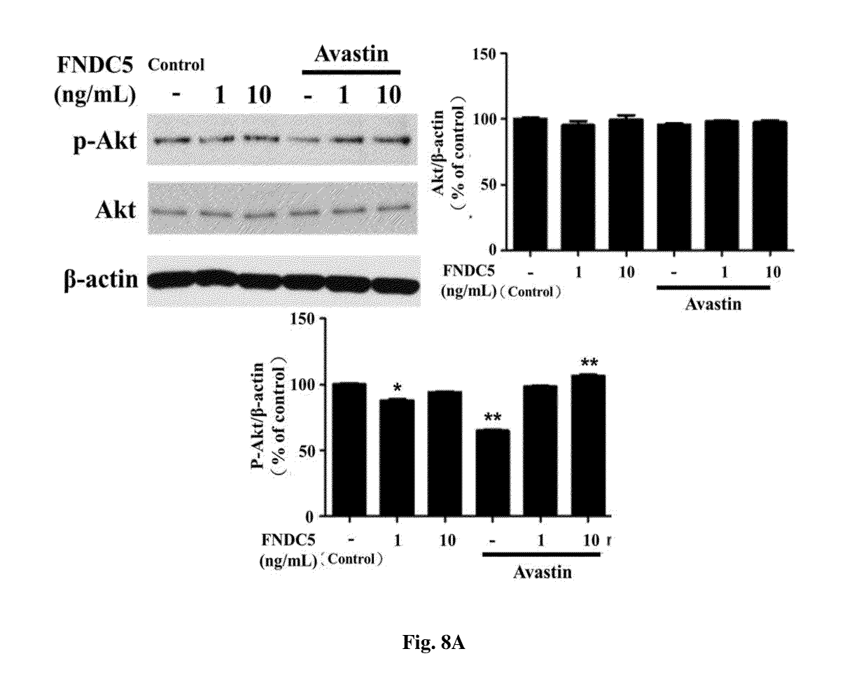

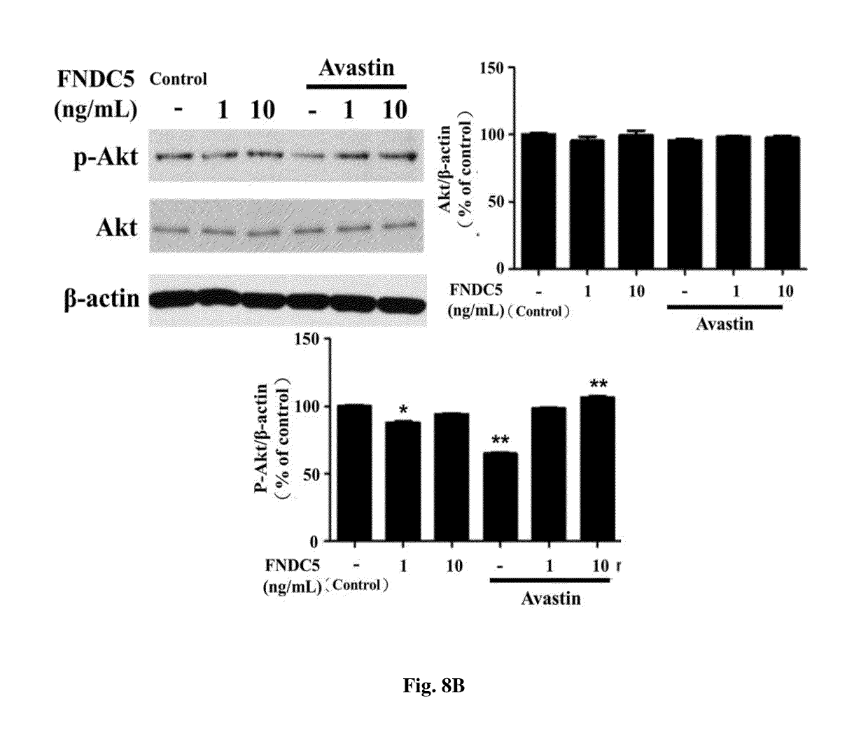

Interestingly, FNDC5 did not promote Akt phosphorylation in endothelial cells by the western blot (FIG. 8A). But FNDC5 (10 ng/mL) treatment could restore the Akt phosphorylation expression when Avastin was used to inhibit VEGF that inhibit the Akt phosphorylation in endothelial cells (FIG. 8B).

### FNDC5 was not Effect on NOS Signal Pathway in Endothelial Cells

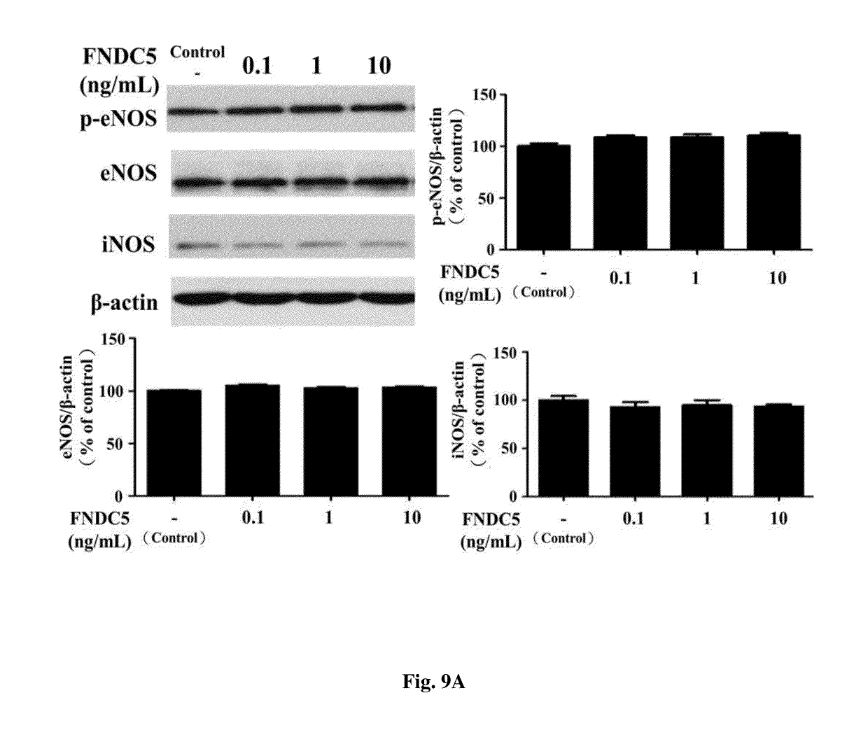

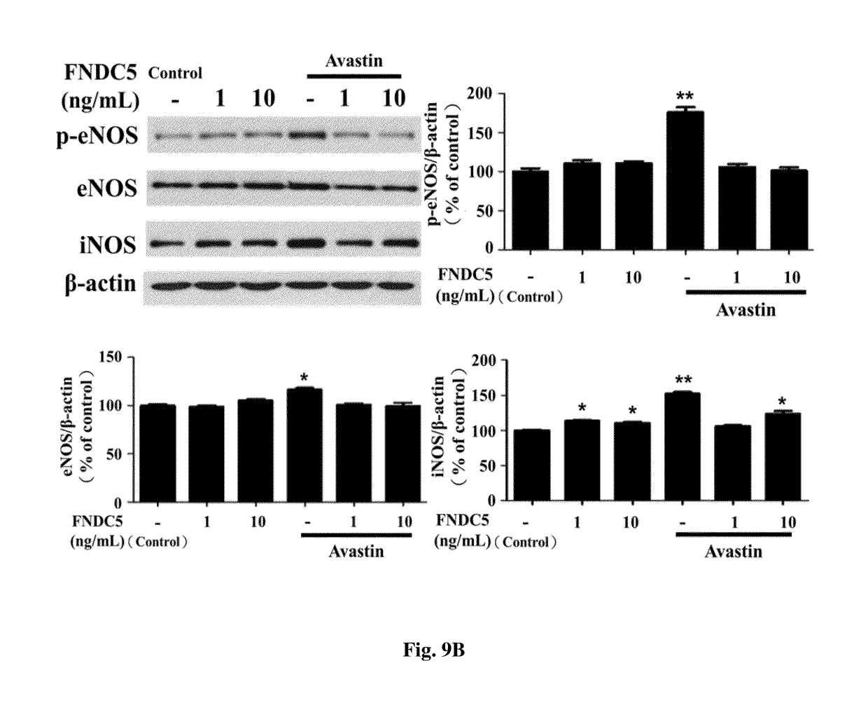

Early studies had demonstrated that VEGF stimulates Akt-mediated endothelial nitric oxide synthase (eNOS) phosphorylation at Ser (serine) 1177, leading to increasing nitric oxide (NO) activity. Moreover, it had been also reported that production of NO in response to fluid shear stress in cultured endothelial cells is controlled by Akt-dependent phosphorylation of eNOS. To evaluate the influence of FNDC5 on NO expression and NOS pathway in the cells, HUVECs were treated with FNDC5 (10 ng/mL) for 24 h and subjected to a Western blot analysis. The Western blot analysis revealed that FNDC5 does not affect the p-eNOS, eNOS and inducible nitric oxide synthase (iNOS) expression in endothelial cells (FIG. 9A). However, after adding Avastin (25 μg/mL), HUVECs were treated with FNDC5 (1 and 10 ng/mL) for 24 h and subjected to a Western blot analysis. FNDC5 treatment could restore the eNOS phosphorylation, eNOS and iNOS expression when Avastin was used to inhibit VEGF activation (FIG. 9B).

### FNDC5 Induced VEGF Expression by NFκB in Endothelial Cells at Transcriptional Level

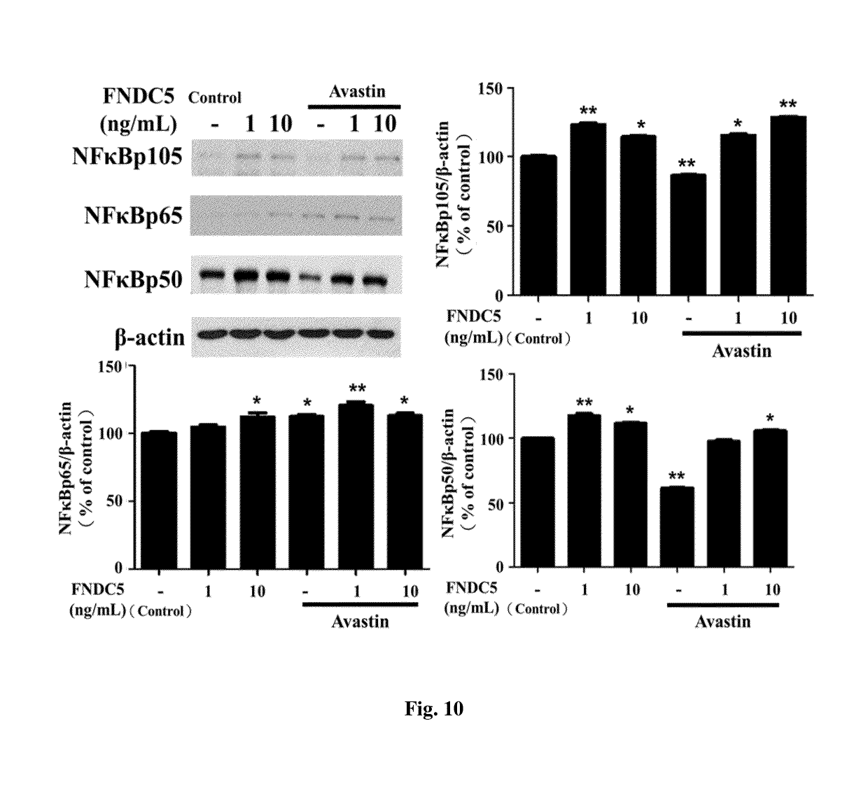

NFκB is a transcription factor responsible for cytokine production, cell survival and the main mediating VEGF in endothelial cells. To evaluate the effect of FNDC5 on NFκB pathway in the cells, the western blot analysis revealed that FNDC5 induced the NFκB expression in endothelial cells (FIG. 10). After adding Avastin (25 μg/mL), HUVECs were treated with FNDC5 (10 ng/mL) for 24 h and subjected to a western blotting analysis. Avastin was used to inhibit VEGF confirmed that FNDC5 (1 and 10 ng/mL) treatment enhance the NFκB p105, NFκB p65 and NFκB p50 protein level expression in endothelial cells (FIG. 10).

### FNDC5 Induces VEGF Expression by HIF-La in Endothelial Cells at Transcriptional Level

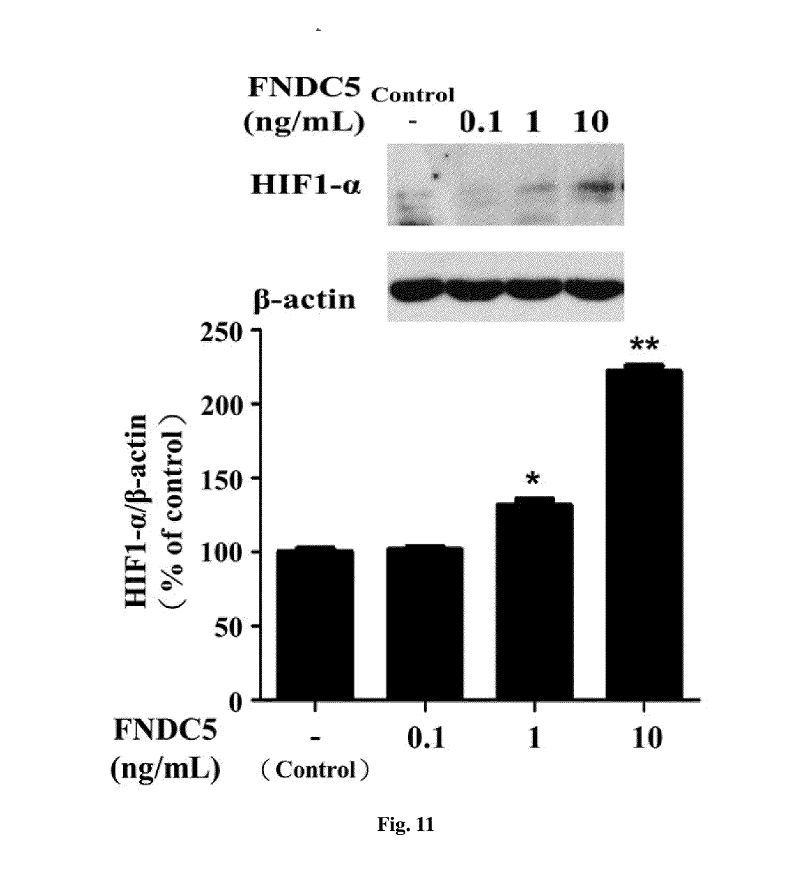

Hypoxia-inducible factor 1 alpha (HIF1-α) is an upstream of VEGF which growing at low oxygen concentrations. To evaluate the effect of FNDC5 on HIF1-α expression, the western blotting analysis revealed that FNDC5 (1 and 10 ng/mL for 24 h) induced the HIF1-α expression in endothelial cells (FIG. 11).

Subsequently, the effects of FNDC5 on the distinct wound healing steps was evaluated, including proliferation, wound healing, migration and tube formation, in cultured endothelial cells.

### Example 4

### Methods

### Proliferation Assay

The endothelial cells were cultured in triplicate at a density 3×10³ cells/well in a 96 well plate and incubated 16 hours. After cells were serum starved for 16 hours, FNDC5 protein was treated in different doses (0.1, 1, 10 ng/mL) and VEGF (positive control, 10 ng/mL) on 1% serum for 24 hours. Cells proliferation assay were performed by MTT assay. The formazan in viable cells were dissolved with 100 μL of dimethyl sulfoxide (DMSO) and determined at 570 nm using ELISA reader.

### Results

### FNDC5 Promoted Proliferation in Endothelial Cells

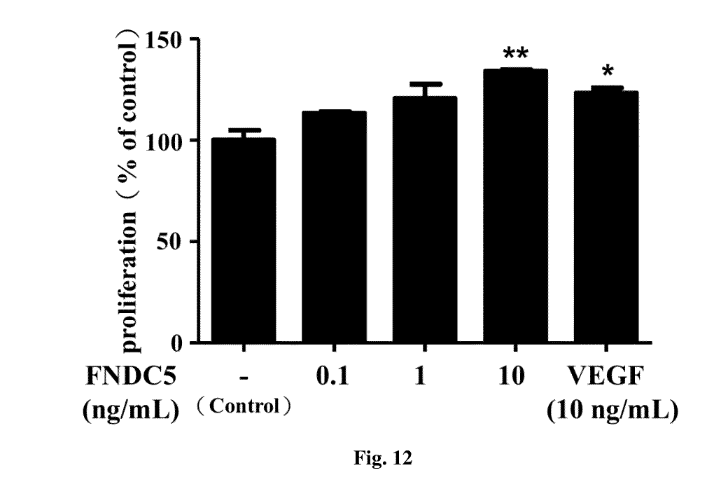

By MTT assay, it was observed that FNDC5 (in physiological concentrations; 0-10 ng/mL) had significantly induced on proliferation of endothelial cells (FIG. 12).

### Example 5

### Methods

### Scratch Wound Healing Assay

The migration of endothelial cells was assessed using a scratch migration assay as described previously (Life Sci. 2008 Jan. 16; 82(3-4):190-204). Briefly, a gap of approximately 1 mm was created in the adherent layer of confluent endothelial cells (in six-well plates) by using a sterile 0.1 mL pipette tip (Gilson, Inc., Middleton, Wis.). After treatment with phosphate buffered saline (PBS), FNDC5 (10 ng/mL), or VEGF (positive control, 10 ng/mL) on 1% serum, the closure extent of the cell-free gap was performed by microscope with digital images system (Olympus; Tokyo, Japan) at different time intervals and measured by NIH Image program.

### Results

### FNDC5 Promoted Healing of Scratch Wound in Endothelial Cells

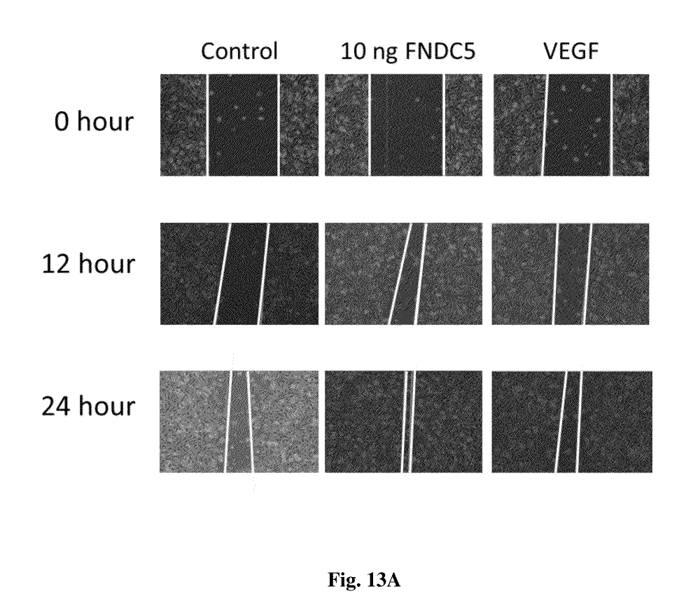

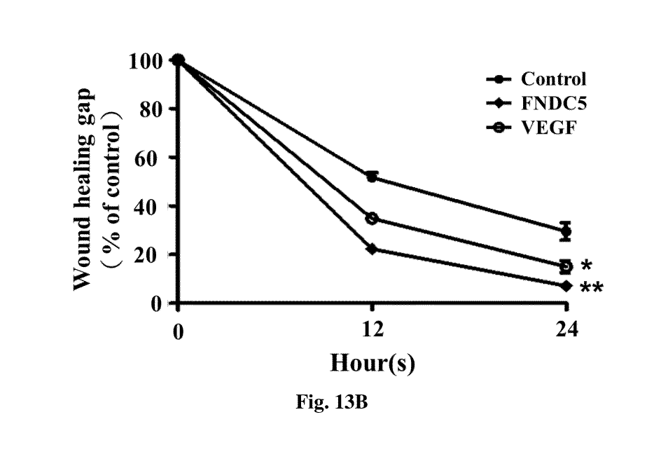

FNDC5 significantly promoted the healing of scratch wound in endothelial cells (FIG. 13A and FIG. 13B).

### Example 6

### Methods

### Migration Assay

Migration assay (Boyden chamber assay) was performed as previously described (Mol Vis. 2009; 15: 1897-1905). In Boyden chamber assay, a polycarbonate filter (8-μm pore size Nucleopore; Costar, Cambridge, Mass.) which was coated with 0.1% gelatin to allow cell adhesion was separated a compartment. The endothelial cells were seeded in triplicate in the upper compartment of the chamber (1.2×10⁵ cells in 400 μL) and treated with FNDC5 (0.1, 1, and 10 ng/mL), irisin (1, 10, and 100 ng/mL), or VEGF (positive control, 10 ng/mL) of various dosages on the polycarbonate membrane. The lower compartment was filled with 200 μL of the DMEM media containing 10% FBS. After incubation for 4 hours in a humidified 5% CO₂ atmosphere chamber at 37° C., the cells on the upper side of the filter were removed to lower side. The migrated cells were fixed in absolute methanol and stained with 10% Giemsa solution (Merck, Germany). Finally, the fixed cells were photographed by microscope with digital images system (Olympus; Tokyo, Japan).

### Results

### FNDC5 Promoted Migration in Endothelial Cells

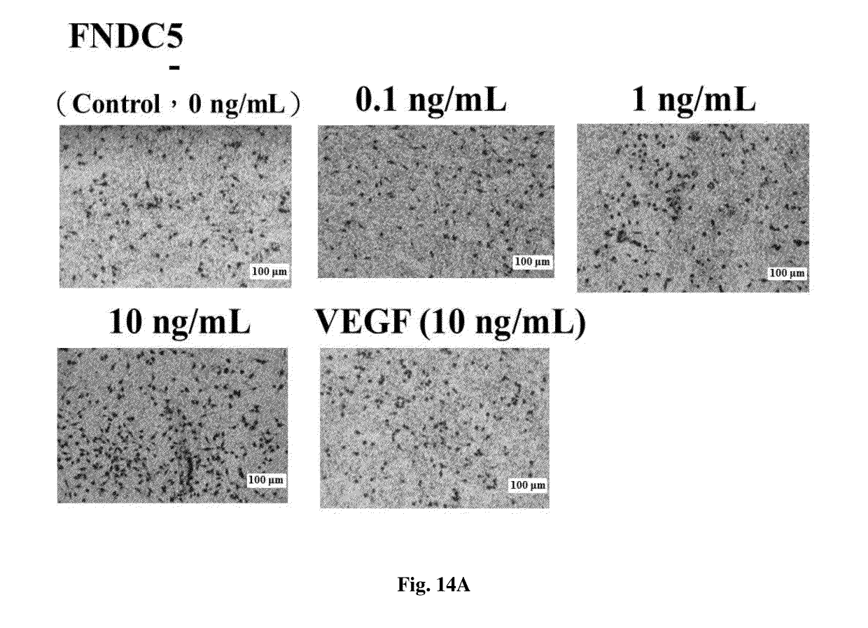

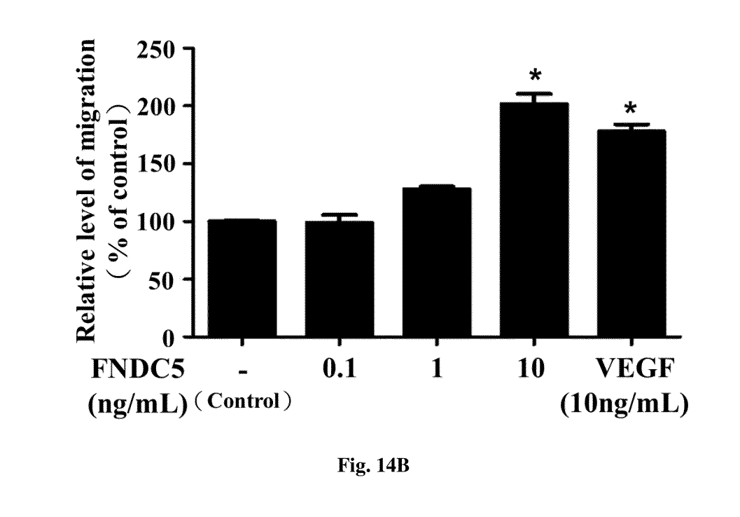

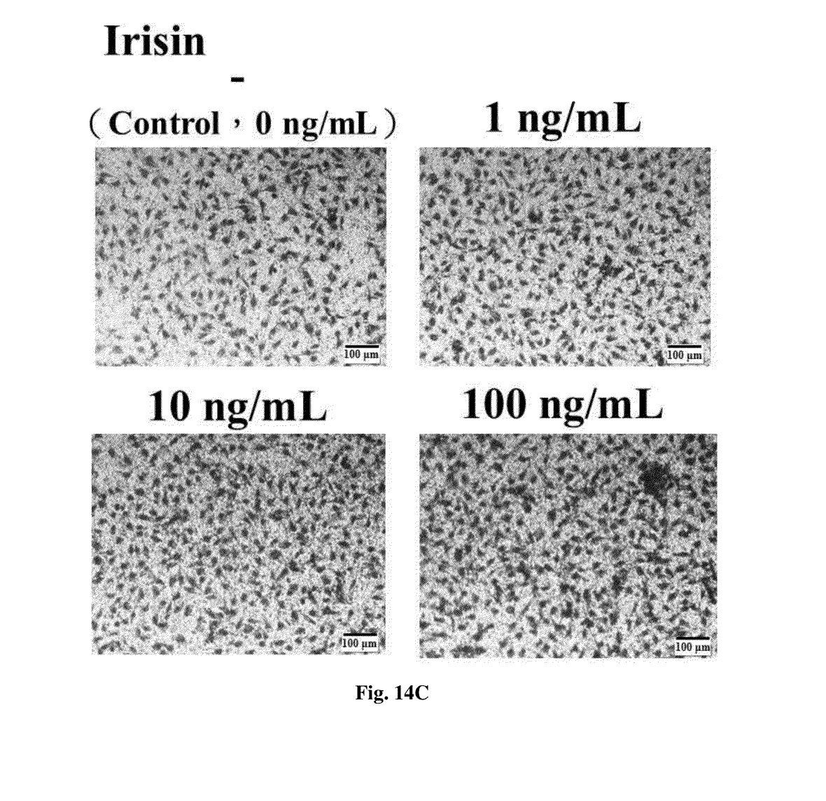

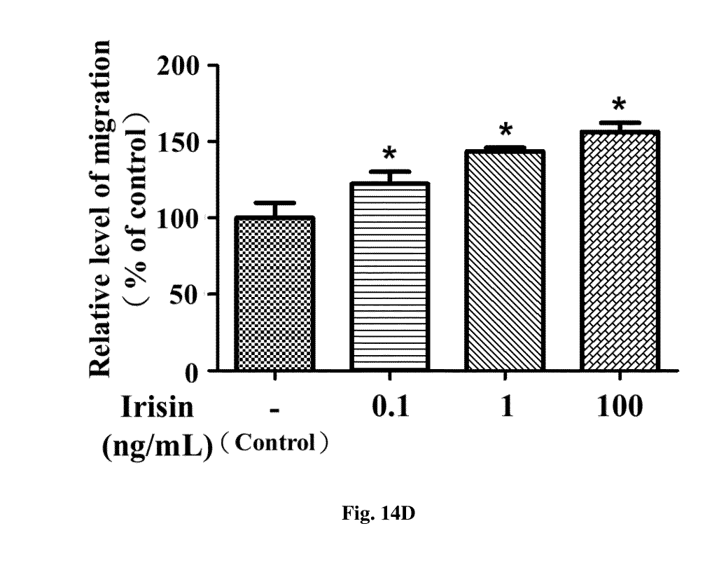

In Boyden chamber assay, FNDC5 (FIG. 14A and FIG. 14B) or irisin (FIG. 14C, and FIG. 14D) enhanced the migration of endothelial cells in dose-dependent manner.

### Example 7

### Methods

### Tube Formation Assay

The tube formation assay was performed as previously described (Atherosclerosis. 2006 June; 186(2):448-57). Matrigel (Becton Dickinson, Bedford, Mass.) was diluted with cold M199 serum-free media to 10 mg/mL. The diluted Matrigel solution was added to 96-well plates (70 μL per well) and allowed to form a gel at 37° C. for 1 hour. The cells suspended (3×10⁴ cells/70 μL per well) in M199 media containing 10% FBS were plated on Matrigel-coated wells and incubated for 6-8 hours at 37° C. in 5% CO₂. After incubation, the endothelial tubes were observed and photographed by microscope with digital images system (Olympus; Tokyo, Japan). Finally, the cells were treated with FNDC5 (0.1, 1, and 10 ng/mL), irisin (10 ng/mL), or VEGF (positive control, 10 ng/mL) and tube formation was quantified by counting the number of rings.

### Results

### FNDC5 Promoted Tube Formation in Endothelial Cells

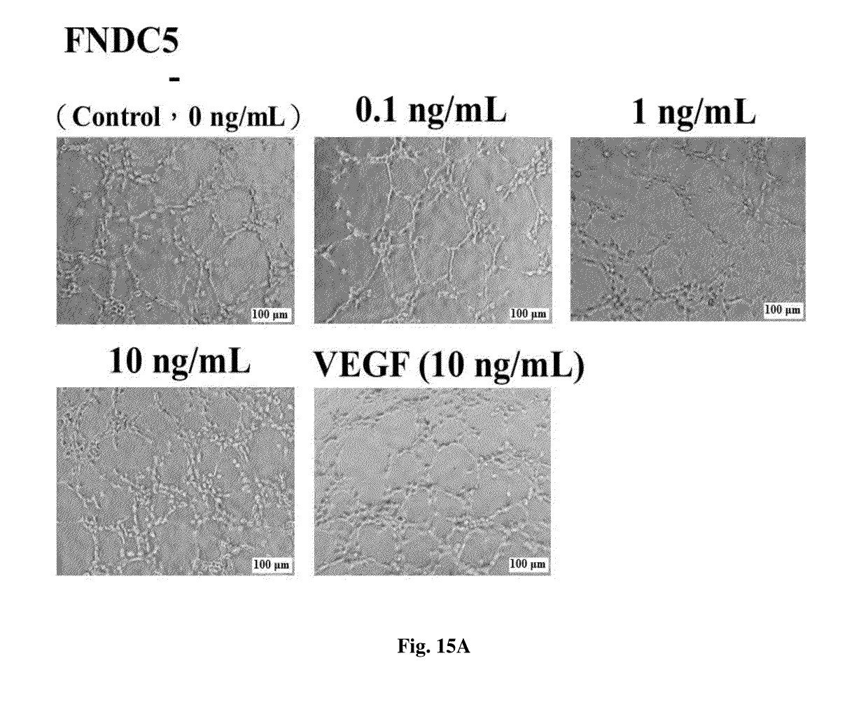

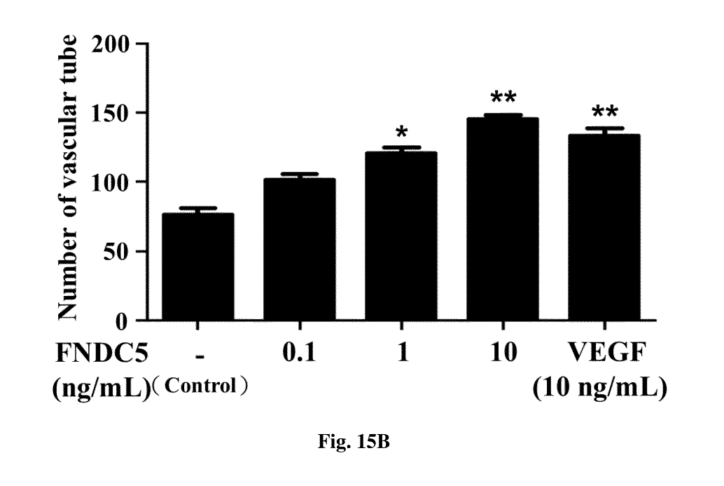

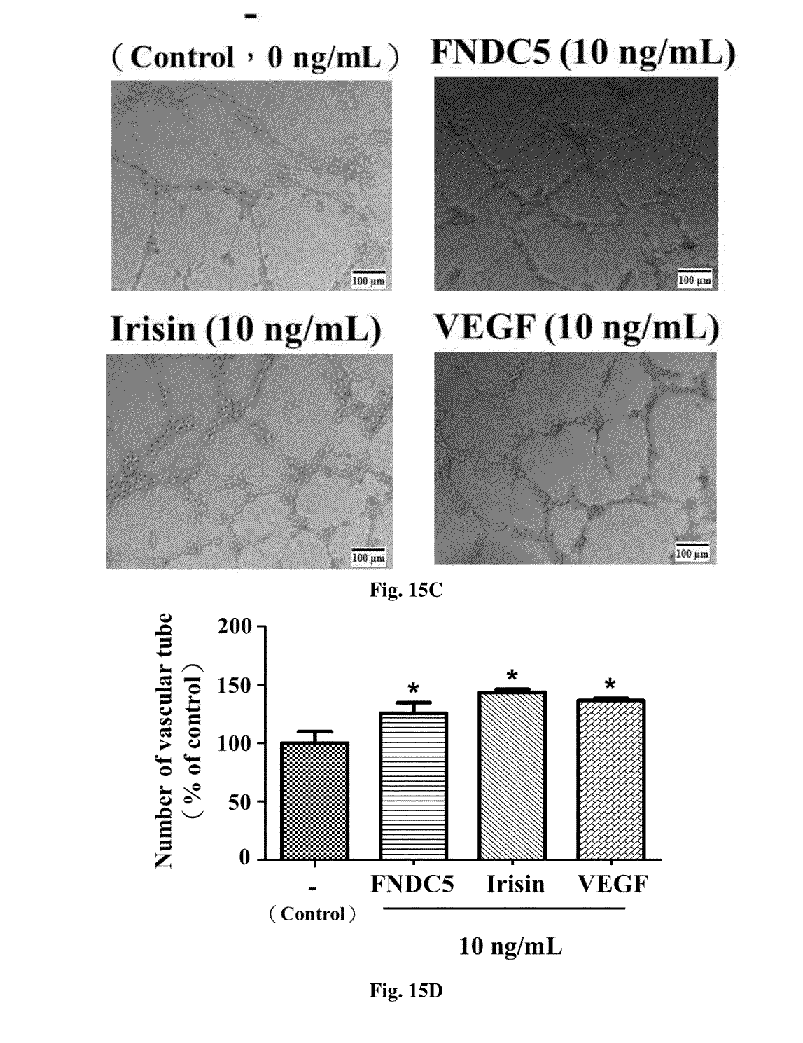

FNDC5 (FIG. 15A, FIG. 15B, FIG. 15C, and FIG. 15D) or irisin (FIG. 15C and FIG. 15D) promoted the formation of tube-like structure of HUVECs.

Taken together, the results of example 4-7 indicated that FNDC5 or irisin promoted proliferation, migration, and tube formation of the cells.

### Example 8

### Methods

### Aortic Ring Assay

Ex vivo, the angiogenesis assay was performed as previously described (Cancer Res. 2007 May 1; 67(9):4328-36). Thoracic aortas were removed from Sprague-Dawley rats (male; 8-week-old) and immediately transferred to a culture dish containing ice-cold serum-free MCDB131 media (Life technologies Ltd., Paisley, Scotland). The peri-aortic fibroadipose tissue was removed with microdissecting forceps and carefully not to damage the aortic wall. Each aortic ring FNDC5 sectioned and extensively rinsed in five subsequent washes of MCDB131 media. Ring-shaped explants of aorta were then embedded in the 1 mL mixtures of Matrigel and MCDB131 (1:1). Then, the aortic rings were polymerized and kept in triplicate at 37° C. in the 24 well culture plates. After polymerization, each well was added with 1 mL of MCDB131 (Life technologies Ltd., Paisley, Scotland) supplemented with 25 mM NaHCO₃, 2.5% rat serum, 1% glutamine, 100 U/mL penicillin, and 100 μg/mL streptomycin and treated with PBS (negative control), FNDC5 (1-100 ng/mL), irisin (1-100 ng/mL), or platelet-derived growth factor (PDGF) to the upper on Matrigel-based embedded aortic ring. The rings were kept at 37° C. in a humidified environment for 7 days and the vascular sprouting was examined by microscope equipped with digital images system (Olympus; Tokyo, Japan). The greatest distance from the aortic ring body to the end of the vascular sprouts (sprout length) was measured by NIH Image program at three distinct points per ring.

### Results

### FNDC5 Affected Angiogenesis Ex Vivo

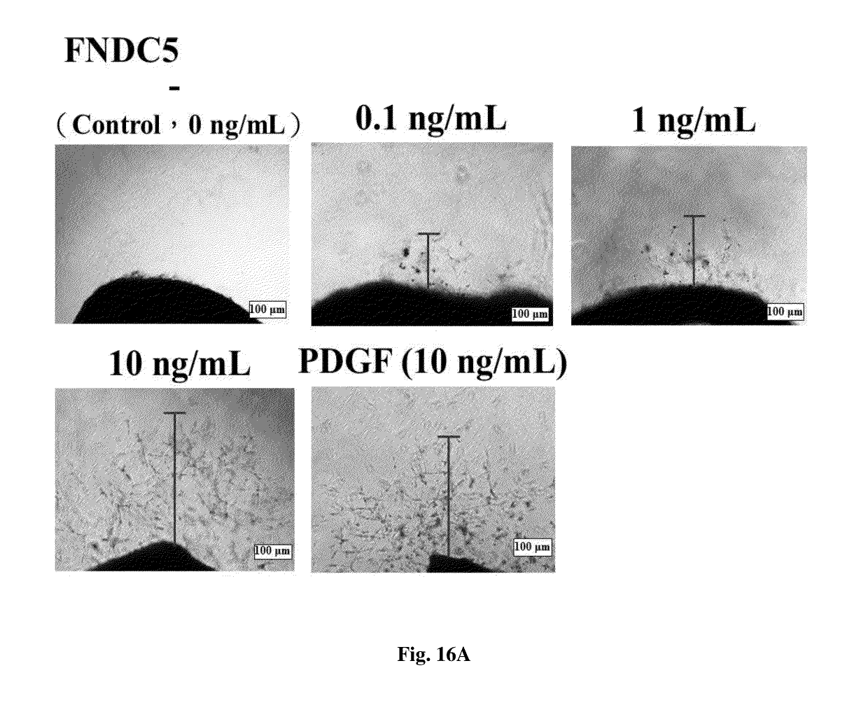

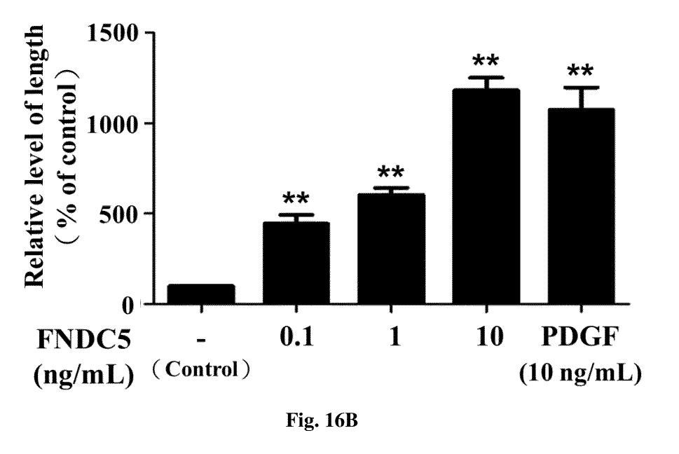

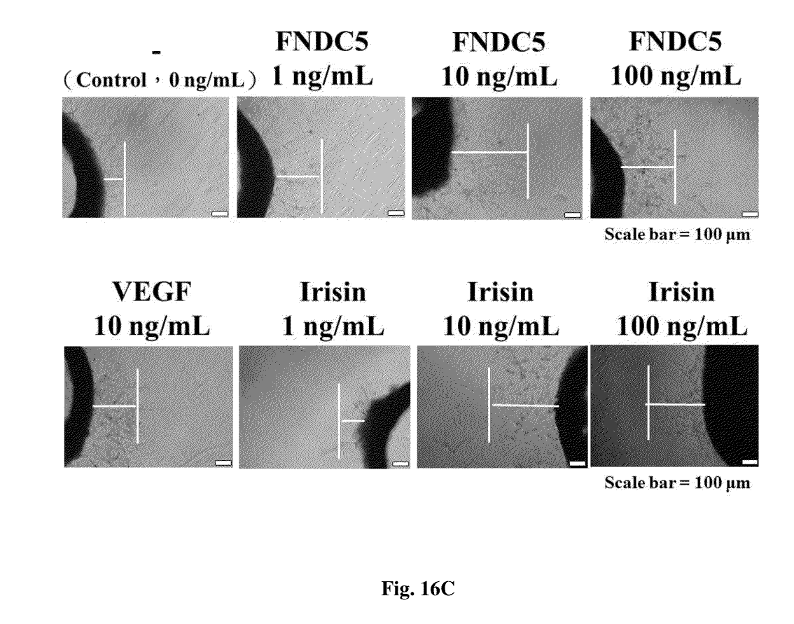

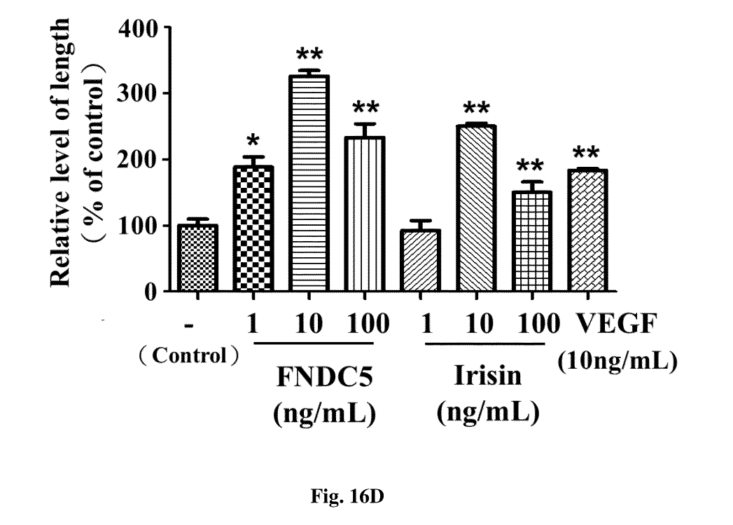

The organotypic aortic rings were used to evaluate the function of FNDC5 on angiogenesis in physiological conditions. It was found that application of FNDC5 (0.1-10 ng/mL) significantly promoted the vessels outgrowth in organotypic aorta cultures (FIG. 16A and FIG. 16B). Moreover, It was also found that application of FNDC5 (1-100 ng/mL) and irisin (1-100 ng/mL) significantly promoted the vessels outgrowth in organotypic aorta cultures (FIG. 16C and FIG. 16D).

### Example 9

### Methods

### Zebrafish Angiogenesis Model

Transgenic Tg (fli-1: EGFP)^{y1} and Tg (fli 1a-nEGFP)^{y7} embryos, in which enhanced Green Fluorescent Proteins (EGFP) is expressed in all endothelial cells of the vasculature were used to monitor the effects of FNDC5 on embryonic angiogenesis (Dev Biol. 2002 Aug. 15; 248(2):307-18). Embryos were treated with 0.003% 1-Phenyl-2-Thiourea (PTU) (Sigma) at six hours post fertilization (hpf) to prevent pigment formation. Zebrafish embryos were generated by natural pair-wise mating and raised at 28° C. in embryo water (0.2 g/l of Instant Ocean Salt in distilled water). Approximately 20 healthy embryos were placed in six-well plates and FNDC5 (10 ng/mL) and VEGF (positive control) were separately added into embryo water at 6 hours post fertilization (hpf). The embryo water containing FNDC5 was replaced daily. At 24 and 48 hpf, the embryos were anesthetized using 0.05% 2-phenoxyethanol in embryo water. The embryos were further observed for blood vessel development, especially in the intersegmental vessels (ISV) and subintestinal vessel plexus (SIV), and subintestinal vessel plexus (SIV), using a microscope with digital images system (Olympus; Tokyo, Japan).

### Results

### FNDC5 Affected Angiogenesis In Vivo

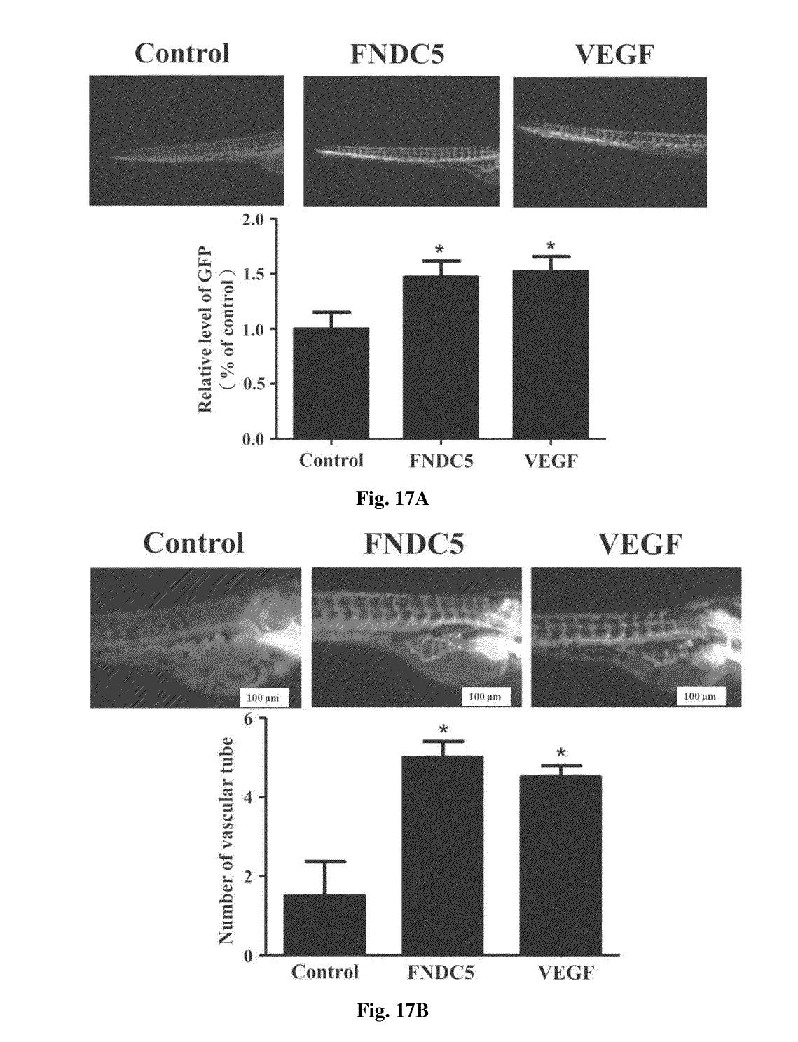

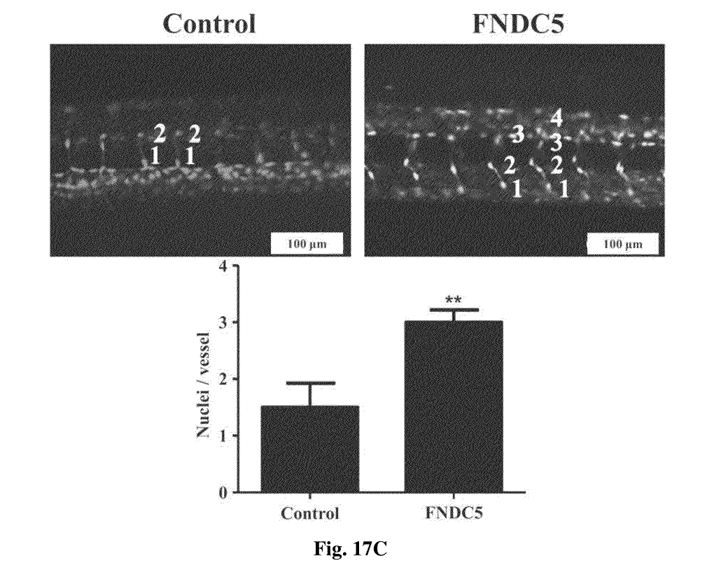

Transgenic zebrafish, Tg (fli-1: EGFP)^{y1}, was used to evaluated the effect of FNDC5 on vascular development. FNDC5 significantly induced the fluorescent intensities of intersegmental vessels (ISV) in zebrafish larva (FIG. 17A). Moreover, FNDC5 promoted the formation of subintestinal vessel plexus (SIV) in zebrafish larva (FIG. 17B). Consistently, FNDC5 induced of endothelial cells in ISV from transgenic zebrafish, Tg (fli 1a-nEGFP)^{y7} (FIG. 17C).

### Example 10

### Methods

### Punch Wound in Diabetic SD Rat Model

Sprague-Dawley rats (SD rats) were purchased from the National Laboratory Animal Center (Taipei, Taiwan), and housed under specific pathogen-free conditions. All animal experiments were carried out under protocols approved by Animal Care and Use Committee of National Sun Yet-Sen University (Kaohsiung, Taiwan). The animals were given free access to food and water and were maintained on a 12 hour light/dark cycle. A subset of the rats was injected intraperitoneally (i.p.) with low dose of STZ (35 mg kg⁻¹). After a week, the rat was anesthetized with pentobarbitone sodium (50 mg/kg body weight) administered intraperitoneally. The animal abdomen was clipped with an electric clipper followed by scrubbing the skin with 70% ethanol and normal saline. A full-thickness circular open wound was generated according to the method reported in the literature (J Am Acad Dermatol. 1997 January; 36(1):53-8), in the abdominal region using a 6-mm sterilized punch biopsy (Stiefel, Germany) in a cranial-caudal direction. A total of four wounds were created on each rat. All freshly created wounds were washed with normal saline before the application of the films. After the wound to be created, FNDC5 and control were randomly applied onto the four wounds of the same rat to eliminate inter-individual differences. The control comprised gauze soaked with normal saline. As twelve rats were used in the study, there were twelve wounds for each treatment. The films and the Control were placed in such a way that the wounds could be completely covered. All the wounds were then covered with non-adherent occlusive gauzes to hold the films in place and further occluded with hypoallergenic adhesive tape. Finally, a bandage was wrapped around the trunk of the animals to protect the dressings. The bandage and the films were changed every days until the wound had completely healed.

### Results

### FNDC5 Enhanced Wound Healing In Vivo

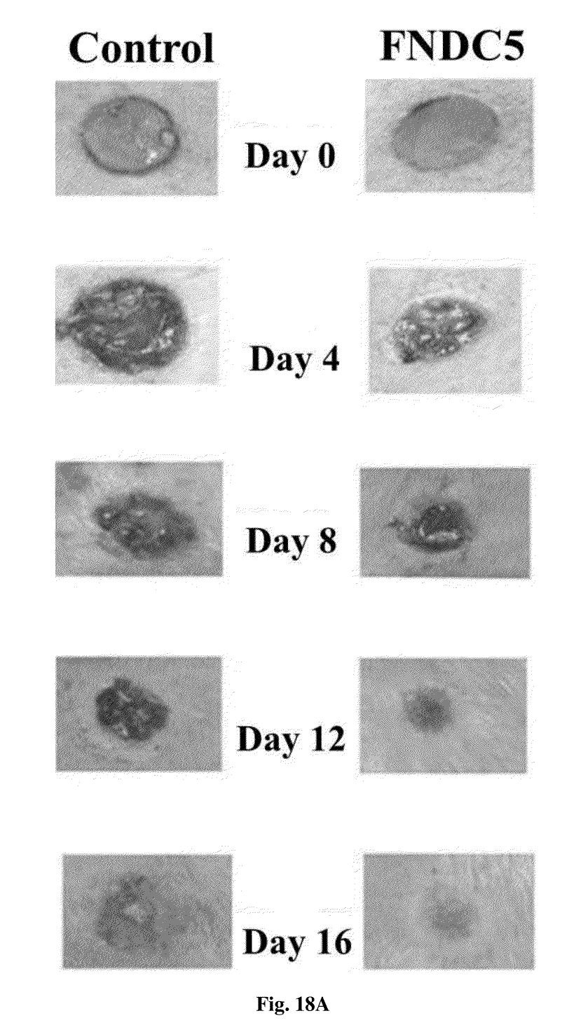

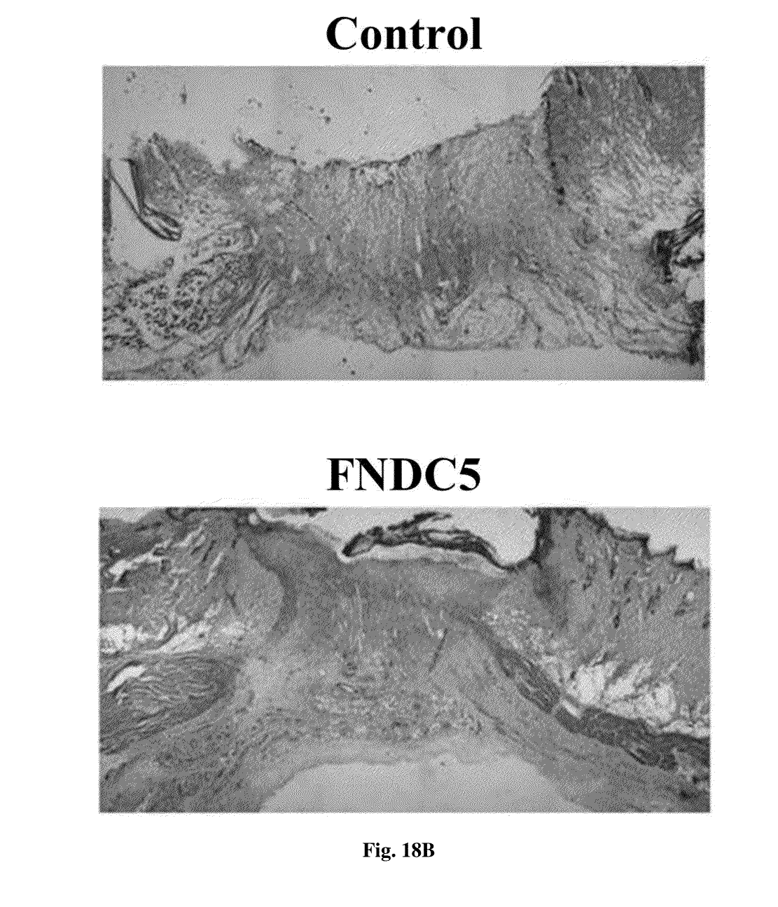

The punch wound diabetic SD rats model was used to evaluate the function of FNDC5 on wound healing in physiological conditions. Two groups of wounded SD rat were treated every two days with phosphate-buffered saline, control group (PBS) and FNDC5 protein (1 mg/mL) alone. In representative wounds (FIG. 18A) and H&E stained images (FIG. 18B) of skin, It was found that application of FNDC5 (1 mg/mL) significantly speeded up the incision wound healing.

One skilled in the art readily appreciates that the present invention is well adapted to carry out the objects and obtain the ends and advantages mentioned, as well as those inherent therein. The composition of the present invention and uses thereof are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method for enhancing wound healing in a subject in need thereof, comprising administering to the subject a composition comprising fibronectin type III domain-containing protein 5 (FNDC5) or its cleaved fragment irisin in an amount effective to enhance wound healing.

2. The method of claim 1, wherein the wound healing is enhanced by stimulation of angiogenesis, cell migration, and cell proliferation in the subject.

3. The method of claim 1, wherein the wound is selected from incisions, lacerations, abrasions, puncture wounds, diabetes ulcers, a burn, blisters, skin tears, donor or graft sites, acnes, contusions, hematoma, crushing injuries or injuries caused by dermabrasion or laser resurfacing.

4. The method of claim 3, wherein the burn results from fire, heat, radiation, electricity, caustic chemicals, or dermatological surgery.

5. The method of claim 1, which is applied to wound healing or reconstructive surgery.

6. The method of claim 1, wherein the composition is in a form selected from gel, cream, paste, lotion, spray, suspension, solution, dispersion salve, hydrogel or ointment formulation.

7. The method of claim 6, wherein the composition is administered to the subject by applying onto skin, injection or electroporation.

8. The method of claim 1, wherein the fibronectin type III domain-containing protein 5 is a recombinant protein expressed by an expression host.

9. The method of claim 9, wherein the expression host is a bacterium, yeast, an insect cell, virus, or a mammalian cell.

10. The method of claim 10, wherein the bacterium is Escherichia coli.
